(19) United States
(12) Patent Application Publication
(10) Pub. No.: US 2019/0168219 A1
(43) Pub. Date: Jun. 6, 2019

(54) SYSTEMS, APPARATUS, AND METHODS FOR INLINE SAMPLE PREPARATION

(71) Applicant: Fluoresentric, Inc., Park City, UT (US)

(72) Inventors: Brian CAPLIN, Park City, UT (US); Brian HICKE, Park City, UT (US); Brandon Doyle LOWE, Park City, UT (US); Jamie PURCELL, Murray, UT (US); Mark POWELSON, Park City, UT (US); Bryson GREEN, Herber City, UT (US); Cameron ORR, Park City, UT (US); Jennifer CAMPBELL, Park City, UT (US); Katelyn KELLY, Park City, UT (US); David OWEN, Park City, UT (US); Michael MCMAHON, Sandy, UT (US)

(21) Appl. No.: 16/031,365

(22) Filed: Jul. 10, 2018

Related U.S. Application Data
(63) Continuation of application No. PCT/US2017/013000, filed on Jan. 11, 2017.
(60) Provisional application No. 62/277,407, filed on Jan. 11, 2016.

(51) Int. Cl. B01L 3/00; C12M 1/12; C12M 3/06; C12M 1/00; C12M 3/00; C12M 1/34

(52) U.S. Cl. CPC B01L 3/502715; C12M 25/10; C12M 23/16; C12M 23/34; C12M 23/42; C12M 23/48; B01L 2200/028; C12M 41/12; B01L 3/563; B01L 2300/18; B01L 2300/168; B01L 2300/0829; C12M 23/52

(57) ABSTRACT

An apparatus includes a sample holder to hold or receive a sample during use. The apparatus also includes a first chamber operably coupled to the sample holder and configured to receive the sample holder. The first chamber includes at least one extraction reagent for extracting nucleic acid from the sample during use to generate a first treated sample. The first chamber and the sample holder collectively define a common longitudinal axis. The apparatus also includes a second chamber operably coupled to the first chamber and disposed along the longitudinal axis. The second chamber includes at least one oligomer for amplifying the nucleic acid to generate a second treated sample.

### CROSS-REFERENCES TO RELATED APPLICATIONS

This application is a continuation of International Patent Application No. PCT/US2017/013000, filed Jan. 11, 2017, and entitled “SYSTEMS, APPARATUS, AND METHODS FOR INLINE SAMPLE PREPARATION,” which claims priority to U.S. Application No. 62/277,407, filed Jan. 11, 2016, and entitled “AN INLINE SAMPLE PREPARATION DEVICE,” the entire disclosure of which are hereby incorporated herein by reference.

### BACKGROUND

Conventionally, physicians have relied on a patient's symptoms to diagnose and treat diseases. Though symptomology has served the medical community well, different diseases, for example bacterial and viral infections, can present similar symptoms but require quite different treatment regimens. The disparity between causative agent and appropriate treatment can lead to incorrect diagnoses and treatment. Furthermore, the problem of bacterial resistance to the small selection of effective antibiotics can be, at least in part, due to lack of positive identification of an infection prior to commencing treatment.

Throughout the past 40 years or more, liquid or plate based culturing techniques have been the standard for detection and identification of bacterial or viral pathogens within a patient sample. Culture based diagnostic techniques typically use a sample that is collected properly from the patient and transported to a well-equipped laboratory with highly skilled laboratory medical technologists to process the sample so as to ensure accurate diagnostic results. The culture model of disease diagnosis can take several days for the result to be available.

Nucleic acid amplification testing technology can be an alternative disease diagnostic method that can offer accurate and sensitive results. Nucleic acid amplification can detect the nucleic acid from almost any organism and diagnose a disease with a high degree of certainty. The biotechnology industry has generally recognized its unique position to improve human health with the prospect of implementing nucleic acid testing for rapid and accurate diagnosis of human disease.

In nucleic acid amplification test, the nucleic acid (e.g., DNA or RNA) directed from a patient for endogenous disease screening or from an appropriate sample site (i.e. nasopharyngeal sample for influenza patients) is usually combined with the nucleic acid amplification reactants and then moved to the reaction vessel and finally to a thermal-cycler where the diagnostic test can be performed. A typical nucleic acid test can include four steps: (i) collect the sample with an appropriate collection device; (ii) extract nucleic acid from sample; (iii) combine genetic material with test ingredients; and (iv) detect the presence, absence, and/or amount of the genetic material of interest. Traditionally, all but the first step are time consuming and are performed by a skilled medical technologist with a fully equipped molecular diagnostic laboratory. For example, in the step (ii), the extraction of a patient sample can be done manually or on a semi- or fully-automated instrument. This step typically involves a specific chemistry or a mechanical operation to break open any bacterial, viral or other cellular organism to release the nucleic acid into solution. It is common for the free floating nucleic acid to be further concentrated and ultimately purified from the sample material. Isolating the nucleic acid is generally captured by attaching a secondary structure, such as magnetic beads, to the nucleic acid then applying a magnetic field or filter to capture the free floating DNA or RNA, and then the nucleic acid is eluted into an amplification compatible storage solution.

These steps can be time consuming and costly due to the number of steps and the level of manual processes and consumable elements in carrying each test. The technology to perform complex genetic based diagnostic tests in locations that are otherwise ill equipped to perform such testing remains a major issue in the biotechnology industry.

A few industry leading companies have worked toward reducing the skill required for nucleic acid testing. These efforts include combination of microfabrication and liquid processing automation or using amplification chemistries that are capable of withstanding less sample pre-processing and thus taking fewer automation steps. However, they still fall short of the level for point of care testing, or bedside testing, which can also be described as medical diagnostic testing at or near the point of care, i.e., at the time and place of patient care. In point of care testing, it is usually desirable for the process of collecting, processing, and analyzing a patient sample to be as error proof as possible.

### SUMMARY

Systems, apparatus, and methods described herein generally relate to sample preparation for diagnostics. The systems, apparatus, and methods described herein more particularly relate to inline sample preparation for nucleic acid amplification.

In some embodiments, an apparatus includes a sample holder configured to hold or receive a sample during use. The apparatus also includes a first chamber operably coupled to the sample holder and configured to receive the sample holder. The first chamber includes at least one extraction reagent for extracting nucleic acid from the sample during use to generate a first treated sample. The first chamber and the sample holder collectively define a common longitudinal axis. The apparatus also includes a second chamber operably coupled to the first chamber and disposed along the longitudinal axis. The second chamber includes at least one oligomer for amplifying the nucleic acid to generate a second treated sample.

In some embodiments, a method includes coupling a sample holder to a first chamber. The sample holder has a sample disposed therein. The sample holder and the first chamber have a common longitudinal axis when coupled. The first chamber includes at least one extraction reagent for extracting nucleic acid from the sample. The method also includes transporting at least a portion of the sample to a second chamber operably coupled to the first chamber and disposed along the common longitudinal axis. The second chamber includes at least one oligomer for amplifying the nucleic acid.

In some embodiments, a method includes receiving a sample using a sample holder in a sample preparation device. The sample preparation device further includes a first chamber operably coupled to a second chamber. The method also in includes disposing the sample holder into the first chamber and placing the sample preparation device into a device. In response to an instruction from a user, the device extracts nucleic acid from the sample using at least one extraction reagent preloaded in the first chamber, transports at least a portion of the sample to the second chamber, and amplifies the nucleic acid using at least one oligomer preloaded within the second chamber.

In some embodiments, a sample preparation device includes a sample holder configured to receive a sample, a first chamber, and a second chamber. The first chamber includes a first compartment to receive the sample holder. The sample holder is removable from the first compartment and hermetically sealing the sample preparation device when placed in the first compartment. The first chamber also includes a second compartment operably coupled to the first compartment and a first valve disposed within the first compartment. The first valve permits fluid flow from the first compartment to the second compartment and to blocks fluid flow from the second compartment to the first compartment. The second compartment draws at least a portion of the sample from the first compartment to the second compartment upon moving away from the first compartment. The first chamber includes at least one extraction reagent preloaded within the first chamber to extract nucleic acid from target microorganism in the sample. The second chamber includes a third compartment operably coupled to the second compartment and a second valve disposed between the second compartment and the third compartment. The second valve permits fluid flow from the second compartment to the third compartment and to blocks fluid flow from the third compartment to the second compartment. The second compartment transports at least a portion of the sample from the second compartment to the third compartment upon moving closer to the first compartment. The second chamber includes at least one oligomer preloaded within the second chamber to amplify the nucleic acid extracted by the at least one extraction reagent.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTIONS OF THE FIGURES

The skilled artisan will understand that the drawings primarily are for illustrative purposes and are not intended to limit the scope of the inventive subject matter described herein. The drawings are not necessarily to scale; in some instances, various aspects of the inventive subject matter disclosed herein may be shown exaggerated or enlarged in the drawings to facilitate an understanding of different features. In the drawings, like reference characters generally refer to like features (e.g., functionally similar and/or structurally similar elements).

FIG. 1 shows a block diagram of an apparatus for inline sample preparation, according to embodiments.

FIG. 2 shows a schematic of device to receive the apparatus shown in FIG. 1 for inline sample preparation, according to embodiments.

FIG. 3 shows an exploded view of a sample preparation device, according to embodiments.

FIG. 4 shows a schematic of a sample amplification and detection chamber that can be used in the apparatus shown in FIGS. 1 and 3, according to embodiments.

FIG. 5A shows an exploded view of a sample preparation device including a spike for sample transportation, according to embodiments.

FIG. 5B shows photos of a sample preparation device and a controller for inline sample preparation, according to embodiments.

FIGS. 6A-6B illustrate methods of inline sample preparation, according to embodiments.

FIGS. 7A-7C illustrates a method of conducting an XCR′ GAS Direct Test using inline sample preparation, according to embodiments.

FIG. 8 illustrates a diagnostic instrument and automated swab processing, according to embodiments.

FIG. 9 shows an example graphical representation of a design for overlapping primer annealing temperatures and template denaturation temperatures, according to some embodiments.

FIG. 10 provides an example illustration of conventional amplification products by real time PCR, according to some embodiments.

FIG. 11 shows an example graph showing high temperature PCR amplification of the same template used in FIG. 10, according to some embodiments.

### DETAILED DESCRIPTION

Overview

To address the challenges in conventional nucleic acid amplification testing technology, aspects disclosed herein are directed to a modular, configurable, and self-contained sample preparation device (SPD) to streamline the nucleic acid testing process, thereby allowing point of care testing of patient samples. In some embodiments, the SPD includes a modular sample holder (also referred to as a sample receiving chamber), which allows the SPD to be readily configurable for any typical sample source (e.g. blood, feces, sputum, and swab, among others). The SPD can include preloaded chemical ingredients to isolate the nucleic acid. Through a series of mechanical movements and based on syringe technology, the SPD combines the nucleic acid testing reagents with the isolated nucleic acid. The amplification chemistry contained within the SPD is accurate to same or higher sensitivity as currently accepted nucleic acid testing chemistries.

Additionally, the amplification step afforded by the combination of the chemistry and the configuration of the reaction chamber can be rapid. Conventional nucleic acid testing chemistries and instruments can take up to one hour for just the amplification step. In contrast, the SPD disclosed herein, when coupled with a diagnostic instrument as described below, is capable of processing the sample, amplifying the nucleic acid, and generating an automated diagnostic result in less than 20 minutes. Accordingly, the SPD offers medical professionals the ability to perform nucleic acid testing at the point of need. The SPD can also guide real-time treatment decisions in a near patient setting.

In some embodiments, the disclosed SPD can be employed as a single-use, disposable, consumable reagent that is compatible with the extreme chain reaction (XCR™) Diagnostic instrument/device. More information on XCR™ technology can be found in PCT Publication No. WO 2016/007914 A1, entitled “DNA AMPLIFICATION TECHNOLOGY,” PCT Application No. PCT/US2016/052335, entitled “APPARATUS, SYSTEMS AND METHODS FOR DYNAMIC FLUX AMPLIFICATION OF SAMPLES,” U.S. Pat. No. 7,838,235, entitled “SYSTEM AND METHOD FOR HIGH RESOLUTION ANALYSIS OF NUCLEIC ACIDS TO DETECT SEQUENCE VARIATIONS,” U.S. Pat. No. 9,139,882, entitled “SYSTEM AND METHOD FOR HIGH RESOLUTION ANALYSIS OF NUCLEIC ACIDS TO DETECT SEQUENCE VARIATIONS,” and U.S. Pat. No. 9,353,408, entitled “DYNAMIC FLUX NUCLEIC ACID SEQUENCE AMPLIFICATION,” the entire disclosure of each is hereby incorporated herein by reference.

The combination of the consumable SPD (e.g., configured for a specific pathogen or assay) and a detection instrument can form a diagnostics platform such as, for example, the XCR™ platform offered by Fluoresentric, Inc. The platform can perform molecular diagnostic testing of a human specimen to diagnose common microbial infections and pathogens. Each SPD can receive a single patient sample and can report a single result before being disposed of. Each SPD also contains preloaded chemical reagents and internal controls to allow fast nucleic acid extraction, amplification, and detection. In this manner, the platform is self-contained because it can carry out clinical diagnosis without addition of ancillary chemical components or reagents or any user manipulation of the device to process the specimens.

Inline Sample Preparation Devices

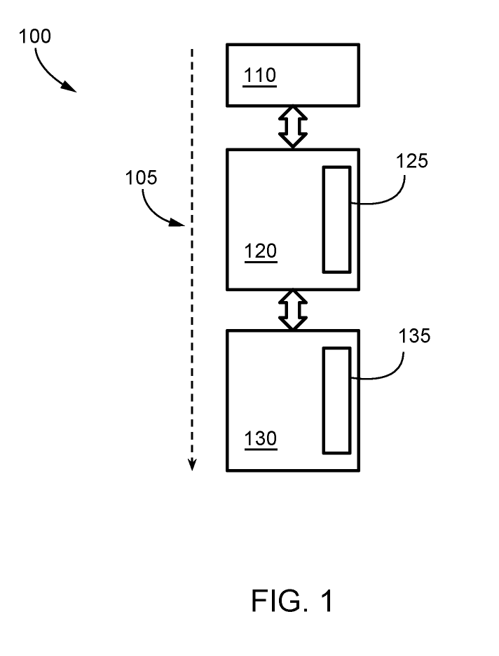

FIG. 1 shows a block diagram of an apparatus 100 for inline sample preparation, according to embodiments. The apparatus 100 includes a sample holder 110 (also sometimes referred to as a “sample receiver”, a “sample input module”, a “sample receiving module”, and variants thereof) configured to hold or receive a sample (also referred to as a test sample) during use. The apparatus 100 also includes a first chamber 120 (also sometimes referred to as a “sample processing chamber”, a “sample processing module”, and variants thereof) operably coupled to the sample holder 110 and configured to receive the sample holder 110. The first chamber 120 includes at least one extraction reagent 125 for extracting nucleic acid from the sample during use to generate a first treated sample. In some embodiments, the first chamber 120 and the sample holder 110 collectively define a common longitudinal axis 105. In some embodiments, the longitudinal axis is also the direction of movement of the sample as the sample undergoes sample extraction, amplification, detection, and other steps. The apparatus 100 also includes a second chamber 130 operably coupled to the first chamber 120 and disposed along the longitudinal axis. In some embodiments, the second chamber 130 includes at least one oligomer 135 for amplifying the nucleic acid to generate a second treated sample.

FIG. 1 shows that the first chamber 120 includes extraction reagent 125 for illustration purposes only. In some embodiments, the extraction reagent 125 can be optional. In this case, sample can be preprocessed to extract nucleic acid before the sample is placed onto the sample holder 110. In some embodiments, the sample can undergo culturing or pre-incubation and then be disposed in the sample holder 110. Similarly, FIG. 1 shows that the second chamber 130 includes oligomer 125 for illustration purposes only. In some embodiments, the amplification of the nucleic acid can be achieved by other methods, such as electrochemical nucleic acid amplification. In some embodiments, the amplification of the nucleic acid can be achieved by methods such as loop mediated isothermal amplification (LAMP), nucleic acid sequence based amplification (NASBA), self-sustained sequence replication (3SR), rolling circle amplification (RCA), strand displacement amplification (SDA), and ligase chain reaction (LCR), among others.

Sample Holder

The sample holder 110 can include any suitable component(s) configured to receive, collect, obtain, acquire, and/or hold a sample to be analyzed. In some embodiments, the sample holder 110 can include a swab that can be employed to acquire the sample, such as a throat culture for example, as well as to hold the sample during use of the apparatus 100. In some embodiments, the sample holder 110 can include a pipette that can be employed to acquire and hold liquid samples. In some embodiments, the sample holder 110 can include a syringe to acquire and hold liquid samples.

In some embodiments, the test sample can be pre-processed prior to being added to the sample holder 110. In some embodiments, the pre-processing can include filtering, concentration, and dilution, among others. In some embodiments, the test sample can be added directly to the sample holder 110 without additional manipulation. In some embodiments, the sample can undergo pre-culturing before being added to the sample holder 110. In some embodiments, the sample can undergo one or more enrichment steps before being added to the sample holder 110. In some embodiments, one or more pre-incubation steps can be performed on the sample before placing the sample onto the sample holder 110.

In some embodiments, the test sample includes, without limitation, blood, serum, plasma, urine, stool, feces, mucous, saliva, sputum, semen, buccal swab, dry swabs, cerebral spinal fluid (CSF), elution/storage media, blood culture, enriched broths from primary culture of human specimen, cultured broths, cells in culture, cell supernatants, cell lysates, and tissue samples.

In some embodiments, the test sample includes environmental material(s), such as (but not limited) surface matter, soil, water, and industrial materials, samples obtained from food products and food ingredients such as dairy items, vegetables, meat, meat by-products, and waste, as well as material obtained from food and dairy processing instruments, apparatus, equipment, disposable, and non-disposable items.

In some embodiments, the sample holder 110 is configurable or customizable for each test sample type. In some embodiments, the sample holder is configured for use with a single test sample type. For example, the extraction reagent 125 and the DNA oligomer 135 preloaded in the apparatus 100 can be configured to extract and amplify, respectively, a predetermined type of nucleic acid.

In some embodiments, the sample holder 110 is configured for use with more than one test sample type. For example, a Group A Streptococcus SPD can have a sample holder 110 configured for use with throat swabs, and a Chlamydia/Gonorrheaa SPD test can have a sample holder 110 configured for either swabs and/or urine. In some embodiments, the sample holder 110 is configured for use with at least two, at least three, at least four, at least five, at least six, at least seven, at least eight, at least nine, at least ten, at least fifteen, or at least twenty test sample types.

In some embodiments, the sample holder 110 is capable of manipulation of a test sample. In some embodiments, the manipulation can improve the homogenization of the test sample. In some embodiments, the manipulation of a test sample includes, without limitation, filtration of the test sample, dilution of the test sample, concentration of high-volume test samples such as blood or urine, or any combinations thereof.

First Chamber

In some embodiments, the first chamber 120 is removably coupled to the sample holder 110. Therefore, a user, such as a physician, can remove the sample holder 110 to acquire a sample from a patient and then places the sample holder 110 back to the first chamber 120 for processing of the acquired sample. In some embodiments, the sample holder 110 can be fitted to the first chamber 120 via barbed fittings. In some embodiments, the sample holder 110 can be fitted to the first chamber 120 via compression fittings. In some embodiments, the sample holder 110 can be fitted to the first chamber 120 via threaded fitting. In some embodiments, the sample holder 110 can be fitted to the first chamber 120 via push-in fittings. In some embodiments, the sample holder 110 can be fitted to the first chamber 120 via O-rings, including tapered O-rings.

In some embodiments, the first chamber 120 can be hermetically sealed by the sample holder 110. For example, the sample holder 110 and the first chamber 120 can form an air-tight seal when they are coupled together. In such embodiments, the first chamber 120 can include a syringe structure having a first compartment and a second compartment that can be translated with respect to the first compartment. Moving the second compartment with respect to the first compartment can draw the sample from the sample holder 110 to first chamber 120. In some embodiments, the first compartment of the first chamber 120 can receive the sample holder 110 and moving the second compartment with respect to the first compartment can draw the sample from the first compartment to the second compartment.

In some embodiments, the syringe structure can include a one-way valve in the first chamber 120 to prevent back flow (e.g., from the first chamber 120 back to the sample holder 110). In some embodiments, the first chamber 120 can further include one or more filters to filter the sample before further processing, such as amplification.

In some embodiments, the first chamber 120 is configured for sample extraction. In some embodiments, the first chamber 120 is configured to extract a target analyte from the test sample. In some embodiments, the target analyte is a cell present in the test sample. In some embodiments, the cell is a cell of non-microbial origin. In some embodiments, the target analyte is a microorganism. In some embodiments, the microorganisms include, but are not limited to, gram positive bacteria, gram negative bacteria, virus particles, parasites, and fungi. In some embodiments, the gram positive bacteria include, without limitation, Actinomyces sp., Bacillus sp., Clostridium sp., Enterococcus sp., Lactobacillus sp., Listeriaceae sp., Pilibacter sp., Staphylococcus sp., and Streptococcus sp. In some embodiments, the gram negative bacteria include, without limitation, Chlamydia sp., Enterobacter sp., Escherichia sp., Flavobacterium sp., Haemophilus sp., Helicobacter sp., Klebsiella sp., Kluyvera sp., Legionella sp., Neisseria sp., Pseudomonas sp., Rickettsia sp., Salmonella sp., Shigella sp., Yersinia sp., Plesiomonas sp., and Vibrio sp. In some embodiments, the gram negative bacterium is Chlamydia trachomatis. In some embodiments, the gram negative bacterium is Neisseria gonorrhoeae. In some embodiments, the virus particle is an RNA virus or a DNA virus. In some embodiments, the virus particle includes, without limitation, Adenoviridae, Baculoviridae, Flaviviridae, Orthomyxoviridae, Papillomaviridae, Paramyxoviridae, Picornaviridae, Reoviridae, Caliciviridae, Astroviridae, Norovirus, Rotavirus, Astrovirus, Sapovirus in stool and food testing, and/or Rhabdoviridae. In certain embodiments, the parasites include, without limitation, Cryptosporidium sp., Entamoeba sp., Giardia sp., Trichomonas sp., and Trypanosome sp. In some embodiments, the parasite is Entamoeba histolytica. In other embodiments, the parasite is Giardia lamblia. In yet other embodiments, the parasite is Trichomonas vaginalis.

In some embodiments, the target analyte is a microorganism found in a test sample. In other embodiments, the target analyte is a microorganism cultured, either from a culture plate or culture broth, in a laboratory under standard microbiological protocols. In certain embodiments, the culture contains only the target microorganism. In other embodiments, the culture contains one or more microorganisms in addition to the target microorganism.

In some embodiments, the first chamber 120 is configured to contain the extraction reagent(s) 125. In certain aspects, the extraction reagent 125 includes, without limitation, buffers, enzymes, detergents, salts, and any combinations thereof.

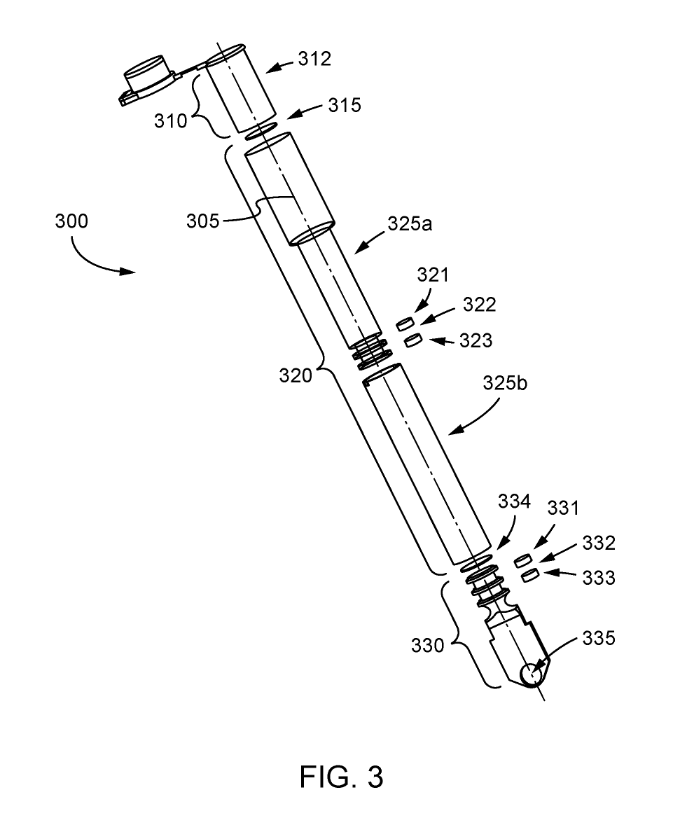

In some embodiments, the extraction reagent 125 is stored in the first chamber 120. In some embodiments, the extraction reagent 125 is stored in the first chamber 120 in a liquid form. In some embodiments, the liquid extraction reagent 125 is stored in a capsule in the first chamber 120. In some embodiments, the liquid extraction reagent 125 is stored in a vessel in the first chamber 120. In some embodiments, the vessel is a capped buffer vessel. In some embodiments, the extraction reagent 125 is contained within a buffer in liquid form within the second chamber in a variety of buffer capsule formats (see, e.g., 312 or 325a in FIG. 3). In some embodiments, the extraction reagent 125 can be stored in a vessel coupled to the sample holder 110 (see, e.g., FIG. 3).

In some embodiments (not shown), the extraction reagent 125 is stored in the second chamber 130 in a solid form. In some embodiments, the extraction reagent 125 is in a powdered form. In some embodiments, the extraction reagent 125 is in a pellet form. In some embodiments, the pellet including the extraction reagent 125 can be disposed in a freestanding form (e.g., stuck in the first chamber 120). In some aspects, the extraction reagent 125 is in a lyophilized form. In other aspects, the extraction reagent 125 is in a dehydrated form. In yet other aspects, the extraction reagent 125 is in a freeze-dried form. In some embodiments, the extraction reagent 125 is dried on to one or more filters (e.g., 321 and 323 in FIG. 3, described in more detail herein) included in the first chamber 120.

In some embodiments, the apparatus 100 can be built such that there is a gap, or inter-filter space, between two filters included in the first chamber 120. This gap can accommodate a thin disk or bead (e.g., 322 in FIG. 3). In some embodiments, the extraction reagent 125 is dried onto the thin disk, or the bead, or both. In some embodiments, the one or more extraction reagent is dried into a pellet or bead and placed within the main sample processing chamber (e.g., 325a in FIG. 3). In some embodiments, combination(s) of these embodiments can also be used to, for example, conduct a test requiring multiple internal controls.

In some embodiments, the extraction reagent 125 extracts one or more target analytes from the test sample. In other embodiments, the extraction reagent 125 additionally extracts nucleic acid from the target analyte. In some embodiments, the nucleic acid includes DNA. In some embodiments, the nucleic acid includes RNA. In some embodiments, the nucleic acid includes a combination of RNA and DNA. In some embodiments, the nucleic acid is substantially the whole genome of a target organism. In some embodiments, the nucleic acid is a portion of the whole genome. In some embodiments, the nucleic acid includes a plasmid. In some embodiments, the nucleic acid includes a vector.

In some embodiments, the extraction reagent 125 can be diluted prior to use. In some embodiments, the extraction reagent 125 can be diluted in water. In some embodiments, the extraction reagent 125 is diluted in buffer. Commonly used buffers include, without limitation, Tris buffer, Tris EDTA (TE) buffer, histidine buffer, acetate buffer, citrate buffer, bicarbonate buffer, phosphate buffer, HEPES buffer, and phosphate buffered saline (PBS). In certain embodiments, the buffer is TE. In other embodiments, the buffer is PBS.

In some embodiments, the extraction reagent 125 is diluted to a concentration of about 5% v/v, about 10% v/v, about 20% v/v, about 30% v/v, about 40% v/v, about 50% v/v, about 60% v/v, about 70% v/v, about 80% v/v, about 90% v/v, or about 95% v/v prior to use. In specific embodiments, the one or more extraction reagent is diluted to a concentration of about 50% v/v.

In some embodiments, the extraction reagent 125 includes one or more detergents. Non-limiting examples of suitable detergents include Tween, NP-40, Polysorbate-80, sodium dodecyl sulfate (SDS), sodium sarcosinate (sarkosyl), lithium dodecyl sulfate, sodium 1-octane sulfonic acid, ethyleneglycoether polymer, tert-octylphenol poly(ethyleneglycoether), and combinations thereof. In specific embodiments, the ethyleneglycoether polymer is Triton-X-100. In some embodiments, the extraction reagent 125 is a detergent-based Mycobuffer Extraction Buffer. In some embodiments, the extraction reagent 125 is an enzymatic-based Achromopeptidase (ACP) Extraction Buffer.

In some embodiments, the extraction reagent 125 includes sodium hydroxide. In some embodiments, the sodium hydroxide is present in the extraction reagent 125 at a concentration of at least about 25 mM. In some embodiments, the extraction reagent 125 is supplemented with 25 mM sodium hydroxide can improve extraction in test samples containing mucous, such as, for example, throat, nasal, vaginal, or cervical swabs. In some embodiments, the extraction reagent 125 comprises a protease. In some embodiments, the protease is a protease from S. girieus. In some embodiments, the presence of the protease improves extraction in test samples containing mucous. In some embodiments, the extraction reagent 125 includes a lytic enzyme. In some embodiments, the lytic enzyme is lysozyme.

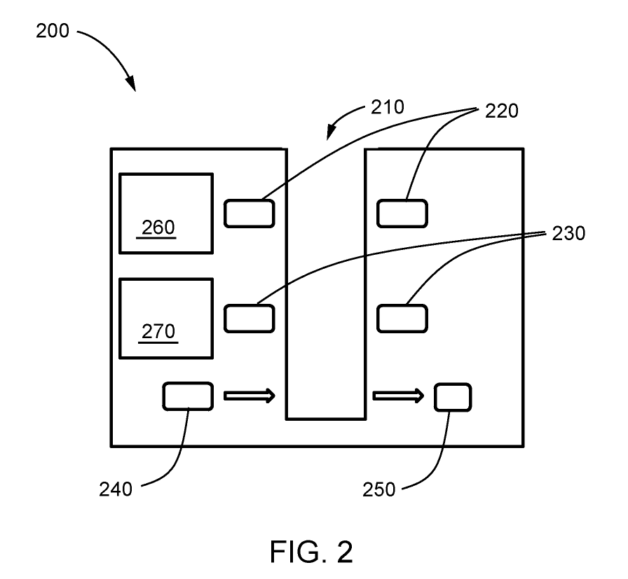

In some embodiments, the extraction process involves a combination of extraction chemistry and heat. In some embodiments, the heat involved in the extraction process can be generated by a separate device that can receive the apparatus 100 (see, e.g., FIG. 2 below).

In some embodiments, a broad temperature range above physiological temperatures can be used. In some embodiments, the temperature ranges from about 38° C. to about 100° C. (e.g., about 38° C. to about 100° C., about 40° C. to about 95° C., about 50° C. to about 95° C., about 60° C. to about 95° C., including any values and sub ranges in between). In some embodiments, a temperature of 95° C. is used for the extraction process. In some embodiments, the extraction process involves treating the test sample at an elevated temperature in the presence of extraction reagent for at least about 2 minutes, at least about 3 minutes, at least about 4 minutes, at least about 5 minutes, at least about 10 minutes, at least about 15 minutes, at least about 30 minutes, or at least about 60 minutes, including all values and sub-ranges in between. In some embodiments, the test sample is treated at an elevated temperature in the presence of extraction reagent for at least about 5 minutes. In some embodiments, a temperature of about 80° C. for about 2-5 minutes is used for the extraction process. In some embodiments, a temperature of 80° C. for 3 minutes is used for the extraction process. In some embodiments, the heating and extraction is carried out in an airtight container (e.g., an airtight container formed by the sample holder 110, the first chamber 120, and the third chamber 130).

In some embodiments, the extraction process involves a combination of extraction chemistry and sonication. In some embodiments, the mechanical contributions of heat and sonication are obtained from a separate device (e.g., the device 200 shown in FIG. 2) or device for which the SPD is designed to coordinate with. In some embodiments, the heating/sonication can be performed on the first chamber 120. In some embodiments, the heating/sonication can be performed on the second chamber 130. In some embodiments, the heating/sonication can be performed on both the first chamber 120 and the second chamber 130.

In some embodiments, the resulting solutions containing extracted target analytes are used directly in the amplification reaction with no further dilution. In some embodiments, the resulting solutions containing extracted target analytes are further diluted prior to the amplification reaction.

In some embodiments, the extraction reagent 125 serves to additionally dilute the test sample. In some embodiments, dilution of the test sample results in dilution of one or more compounds present in the test sample. In some embodiments, the one or more compounds are biological compounds. In some embodiments, the one or more compounds are synthetic compounds. In some embodiments, said synthetic compounds are obtained from enriched broths from primary culture of a test sample. In some embodiments, the one or more compounds are large particulate matter. In some embodiments, the one or more compounds are inhibitory to test sample processing by the first chamber 120. In some embodiments, the one or more compounds interfere with downstream fluidics. In some embodiments, the one or more compounds interfere with downstream amplification process. In some embodiments, dilution of the one or more compounds leads to more efficient test sample processing by the second chamber 120. In some embodiments, the extraction reagent 125 serves to simultaneously dilute the test sample and extract the target analyte from the test sample.

In some embodiments, the extraction reagent 125 is used to dilute the test sample at least about 0.1 fold, at least about 0.2-fold, at least about 0.3-fold, at least about 0.4-fold, at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, or at least about 20-fold, including all values and sub-ranges in between. In some embodiments, the test sample is diluted 10-fold with the extraction reagent 125.

In some embodiments, the test sample is not diluted. In some embodiments, the extraction reagent 125 is dried into the first chamber and completely rehydrated with the test sample. As a result, no dilution of the test sample occurs. In some embodiments, test samples that are not diluted include high-volume samples such as blood or urine.

In some embodiments, the first chamber 120 is configured to affect target enrichment. In some embodiments, the first chamber 120 includes compartments where test samples are seeded into buffers or broths that encourage growth of microorganisms. In some embodiments, the test sample is incubated within the apparatus 100 for some pre-defined amount of time and at a pre-defined temperature that promotes growth of microorganisms. In some embodiments, this method results in concentration of dilute test samples into a smaller volume. In some embodiments, this method results in enrichment of microorganisms within the concentrated solution for the target organisms prior to extraction. In some embodiments, such target enrichment leads to increasing the amount of template available for later downstream applications.

Sample Processing Control (SPC)

In some embodiments, the first chamber 120 contains a sample processing control (SPC). In some embodiments, the SPC has an extraction profile(s) (which can refer to procedures to extract nucleic acid from a sample) similar to the target analyte. Using SPC(s) can increase the reliability of testing results. In some embodiments, the SPC is processed in an identical manner to the target analyte (e.g., subject to extraction, amplification, and detection).

In some embodiments, the SPC is introduced into the test sample prior to analysis. In some embodiments, the SPC is introduced into the apparatus 100 simultaneously with the test sample. In some embodiments, the SPC is introduced into the apparatus 100 prior to the introduction of the test sample. In some embodiments, the SPC is provided in the apparatus 100.

In some embodiments, the SPC includes a whole microorganism. In some embodiments, the SPC includes a nucleic acid. In some embodiments, the SPC includes a naturally occurring nucleic acid. In some embodiments, the SPC includes a synthetic nucleic acid. In some embodiments, detection of the SPC within a pre-defined, expected range indicates that all of the sample processing and amplification reagents and steps are functional and therefore validate the test.

In some embodiments, the SPC can be stored in the sample holder 110 (or the buffer capsule 312 in the sample holder 310 shown in FIG. 3). In some embodiments, the SPC can be stored as a liquid (e.g., in the capsule 312 in FIG. 3). In some embodiments, the SPC can be stored in dried form (e.g., on the filters 321, 323, 331, or 333 in FIG. 3, or in the inter-filter space 322 and/or 332 in FIG. 3).

In some embodiments, the SPC can be stored in the first chamber 120. In some embodiments, the SPC can be stored in the first chamber 120 in a liquid form. In some embodiments, the liquid SPC can be stored in a capsule in the first chamber 120. In some embodiments, the liquid SPC can be stored in a vessel in the first chamber 120. In some embodiments, the vessel is a capped buffer vessel. In some embodiments, the SPC is contained within a buffer in liquid form within the first chamber 120 in a variety of buffer capsule formats (e.g., 312 and/or 325a in FIG. 3).

In some embodiments, the SPC can be stored in the first chamber 120 in a solid form. In some embodiments, the SPC can be dried on to one or both filters (e.g., 321 and 323 in FIG. 3) present in the first chamber 120. In some embodiments, the apparatus 100 can be built such that there is a gap, or inter-filter space, between the two filters that can accommodate a thin disk or bead (e.g., the disk322 in FIG. 3). In some embodiments, the SPC can be dried onto the thin disk, or the bead, or both. In some embodiments, the SPC is dried into a pellet or bead and placed within the first chamber 120 (e.g., the bead 325a in FIG. 3). Any combination of these embodiments is also available if it is desirable for the test to have multiple internal controls.

In some embodiments, the SPC can be stored on any filter (e.g., filters 321, 323, 331, or 323 in FIG. 3). In some embodiments, the SPC can be stored in any inter-filter spaces (e.g., 322 or 332 in FIG. 3).

Second Chamber

In some embodiments, the second chamber 130 includes a compartment in which nucleic acid amplification and detection occurs. In some embodiments, the oligomer 135 includes DNA oligomer. In some embodiments, the oligomer 135 includes RNA oligomer. In some embodiments, detection of nucleic acid can be carried out via fluorescence-based detection. In some embodiments, the nucleic acid is labelled with a radioisotope, and the detection of nucleic acid can be carried out via radioisotope-based detection. In other embodiments, the nucleic acid is labelled with an enzyme, and the detection of the nucleic acid can be carried out via enzyme-based detection.

In some embodiments, the enzyme-based detection involves horse radish peroxidase (HRP)-based detection. In some embodiments, the second chamber 130 contains all of the chemical reagents for the amplification chemistry and fluorescent detection. In some embodiments, the chemical reagents for the amplification chemistry and fluorescent detection include DNA oligomers (e.g., 135) that serve as: 1) amplification primers for the target analyte(s) and the SPC, and 2) tagged probes that facilitate detection of amplified target analyte(s) (including the SPC). In some embodiments, the diagnostic assays evaluate and distinguish multiple pathogens.

In some embodiments, the assays include multiple oligonucleotide sets, each of which can be unique to a single target in addition to the SPC. For example, in the previously mentioned Chlamydia/Gonorrhoeae Assay the second chamber 130 of the Chlamydia/Gonorrhoeae SPC can be configured to contain amplification and detection oligonucleotides for both a Chlamydia target, a Gonorrhoeae target, and an SPC, thereby constituting a three-target (or triplex) amplification reaction.

In some embodiments, the second chamber 130 can be configured to constitute a single-target amplification reaction. In some embodiments, the second chamber 130 can be configured to constitute more than one-target amplification reaction. In some embodiments, the second chamber 130 can be configured to constitute at one-target, least two-target, at least three-target, at least four-target, at least five-target, at least six-target, at least seven-target, at least eight-target, at least nine-target, at least ten-target, at least fifteen-target, or at least twenty-target amplification reaction. In some embodiments, the second chamber 130 can be configured to constitute at least two-target amplification reaction.

In some embodiments, the second chamber 130 can be configured to detect one target analyte in a test sample. In some embodiments, the second chamber 130 can be configured to detect more than one target analyte in a test sample. In some embodiments, the second chamber 130 can be configured to detect at least one, at least two, at least three, at least four, at least five, at least six, at least seven, at least eight, at least nine, at least ten, at least fifteen, or at least twenty target analytes in a test sample. In some embodiments, the second chamber 130 can be configured to detect at least two target analytes in a test sample.

In some embodiments, the second chamber 130 can include multiple, well-defined locations for holding the amplification chemistry (including the oligomer 135). These locations are described in more detail with reference to FIG. 3.

In some embodiments, amplification of nucleic acids targets includes thermal cycling of the chemical reagents and microorganism template within second chamber 130. In some embodiments, thermal cycling capabilities are provided by the heating/cooling system within the diagnostic instrument (e.g., 200 shown in FIG. 2). In some embodiments, detection of amplified nucleic acids and consequential diagnosis of pathogen, includes excitation and specific fluorescent detection of fluorophores. In some embodiments, fluorescent excitation, detection, and signal processing capabilities are provided by the optics systems within the diagnostic instrument (e.g., 200 shown in FIG. 2). In some embodiments, other detection methods, such as electro-chemical detection, radiometric detection, or colorimetric read-outs can also be carried out by the diagnostic instrument using appropriate excitation and detection components.

Systems for Inline Sample Preparation

FIG. 2 shows a schematic of a device 200 that can be used together with the apparatus 100 shown in FIG. 1 to form a system for inline sample preparation. The device 200 can receive the apparatus 100 and process the sample contained in the apparatus 100. The processing can include, for example, extraction, amplification, and detection of nucleic acid in the sample.

The device 200 (also referred to as a diagnostic device or a diagnostic instrument) includes a receptacle 210 to receive an apparatus (e.g., an SPD, such as the apparatus 100 in FIG. 1) and hold the apparatus in place during use. The device 200 also includes a motor 220 configured to make mechanical movement of the SPD. For example, the SPD can include two compartments (e.g., the first compartment 325a and the second compartment 325b shown in FIG. 3) to form a syringe structure so as to draw the sample contained in the SPD through different steps of processing. The motor 220 can then move the two compartments with respect to each other so as to transport the sample within the SPD.

The device 200 also includes a heater 230 to thermo-cycle the sample in the SPD so as to amplify the nucleic acid contained in the sample. In some embodiments, the heater 230 can be configured to generate a temperature profile for XCR™ processes. Examples of temperature profiles for XCR™ are described with more details below. In some embodiments, the heater 230 can be configured to generate a temperature profile for conventional PCR processes. In some embodiments, the heater 230 can be configured for other purposes, such as maintaining the sample at a constant temperature for storage.

FIG. 2 shows one heater 230 for illustration purposes only. In some embodiments, the heater 230 can include multiple heaters. For example, one heater can be used for thermal-cycling and another heater can be used for thermal convection. In some embodiments, more than two heaters can be used for thermal-cycling, convection, incubating, and/or storage, among others.

The device 200 further includes a light source 240 configured to illuminate the SPD (e.g., the second chamber 130). A detector 250 is also included in the device 200 to detect any fluorescent light emitted by the sample in response to the illumination of the light source 240. This fluorescence can be used to determine the presence or absence of a target nucleic acid. In some embodiments, one or more optical filters (not shown in FIG. 2) can be included in front of the detector 250 to filter out unwanted radiation, to select one or more wavelength for selective transmission, and facilitate detection. In some embodiments, the light source 240 can be replaced by an electrode to carry out electrochemical detection.

In some embodiments, the device 200 can be manual. For example, a user can control the motor 220 to transport the sample within the SPD. The user can also control the heater 230 to implement the XCR™ process. The user can further control the light source 240 to illuminate the SPD and then control the detector 250 to take data.

In some embodiments, one or more aspects of the device 200 can be automated. For example, the motor 220 can be automated. In some embodiments, the device 200 can include a memory 260 to store process-executable instructions and a processor 270 operably coupled to the memory 260 to control operation of one or more aspects of the device 200. For example, upon execution of the processor-executable instructions, the processor 270 can be configured to control the motor 220 to transport the sample within the SPD. The processor 270 can also be configured to control operation of the heater 230 to thermos-cycle the sample so as to amplify the nucleic acid, such as via, for example, extreme chain reaction (XCR™). The processor 270 can be further configured to control the light source 250 to illuminate the sample and to control the detector 260 to detect nucleic acid via fluorescence.

Sample Preparation Devices Based on Syringe Structure

FIG. 3 shows a schematic of an SPD 300 based on syringe structure to transport samples within the SPD 300, according to example embodiments. The SPD 300 includes a sample holder 310, a first chamber 320, and a second chamber 330. The sample holder 310 further includes a buffer capsule 312 to contain extraction reagent(s) and a first membrane 315. During use, a user and/or another device (not shown) can pierce the first membrane 315 to release the extraction reagent(s) so as to extract nucleic acid in the sample.

The first chamber 320 includes a first compartment 325a and a second compartment 325b. Two filters 321 and 323 are disposed between the first compartment 325a and the second compartment 325b. The two filters 321 and 323 also define an inter-filter space 322, which can be used to store extraction reagent(s) as described above.

In some embodiments, the two compartments 325a and 325b can be translated with respect to each other so as to draw the sample from the first compartment 325a to the second compartment 325b. To this end, an one-way valve (not shown in FIG. 3) can be included in the first compartment 325a. In this case, moving the second compartment 325b away from the first compartment 325a can draw the sample from the first compartment 325a to the second compartment 325b.

The second chamber 330 includes a third compartment 335, where nucleic acid in the sample can be amplified and/or detected. A second membrane 334 and two filters 331, 333 are disposed between the second compartment 325b and the third compartment 335. The filters 331, 333 define an inter-filter space 332, which can be used to, for example, store extraction reagent or SPC(s).

In some embodiments, the second membrane 334 can be pierced so as to transport the sample from the second compartment 325b to the third compartment 335. In some embodiments, the piercing can be achieved by thermal pressure. For example, a heater (e.g., 230 in FIG. 2) can be used to heat the sample, which in turn can break the second membrane 334. In some embodiments, the second membrane 334 may be replaced by another one-way valve. In this case, moving the second compartment 325b toward the first compartment 325a can also transport the sample from the second compartment 325b to the third compartment 335. Alternatively, the second one-way value can be included in addition to the second membrane 334. Moving the second compartment 325b toward the first compartment 325a can break the second membrane 334 as well as force open the second one-way valve, thereby transporting the sample from the second compartment 325b to the third compartment 335.

Multiple locations in the second chamber 330 can be used to store chemistries (including SPC) for amplifying and/or detecting nucleic acid in the sample. In some embodiments, the chemistries can be stored on one or both the filters 331 and/or 333. In some embodiments, the chemistry or SPC can be dried onto either of these filters 331 and/or 333. In some embodiments, the inter-filter space 333 between the two filters 331 and 333 can accommodate a thin disk or bead, which can have amplification/detection chemistry or SPC dried onto them. In some embodiments, the chemistry can be dried directly into the third compartment 335. Any combination of these embodiments is also available if a test involves multiple internal controls.

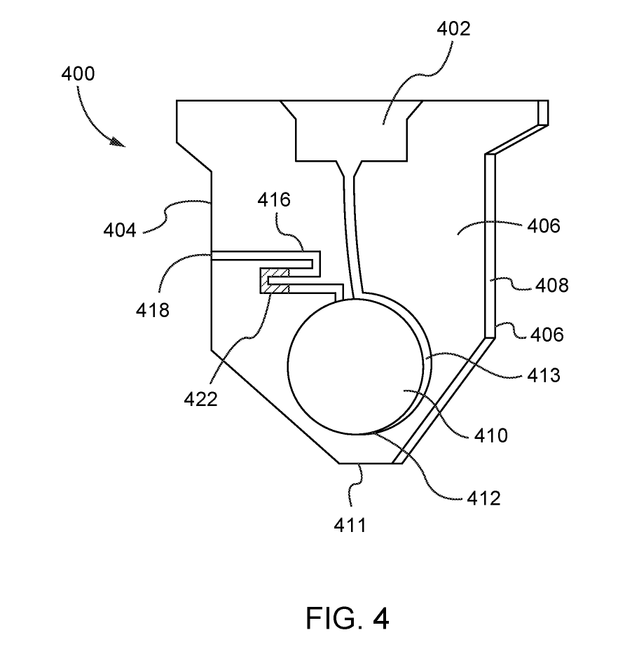

FIG. 4 shows a schematic of a chamber 400 that can be used as the second chamber 330 in FIG. 3 and/or the second chamber 130 in FIG. 1. The chamber 400 includes two major walls 406 (one major wall is not shown in the front view in FIG. 4) and a minor wall 408. The exterior surface 404 of the chamber 400 can have various shapes such as a polygon, a sphere, and a cubic, among others. The interior of the chamber 400 can define a compartment 410, also referred to as the reaction chamber 410, where nucleic acid(s) in the sample can be amplified and/or detected. The chamber 400 includes a closed loop with continuous curves. In some embodiments, different portions of the curve include varying degrees of arc. The bottom of the chamber 400 can have a substantially flat surface 411.

The chamber 400 includes a first port 412, which is in fluid communication with an oligo chamber 402 via a channel 413. The oligo chamber 402 can be used to store oligomers for nucleic acid amplification. The chamber 400 also includes an exit channel 416 coupled with an external port 418 that vents to the atmosphere.

The exit channel 416 is sized and shaped to hold a hydrophilic substance, such as a powdered hydro gel or a hydrophilic yarn 422 that is chemically formulated to expand when contracted by a liquid. As liquid is forced into the reaction chamber 410, the hydrophilic yarn 422 can force air out of the reaction chamber 410 via the exit channel 416. As liquid reaches the top of the reaction chamber 410, the liquid exits out the exit channel 416. As the liquid contacts the hydrophilic yarn 422, the hydrophilic yarn 422 can absorb the liquid and swells until the hydrophilic yarn 422 effectively seals the exit channel 316 and blocks the further egress of more liquid. In some embodiments, the exit channel 416 can be configured to hold one or more hydrophobic vents. In some embodiments, the hydrophobic vents can include Polytetrafluoroethylene (PTFE). In some embodiments, the hydrophobic vents can also include Activated carbon adsorbent that can provide relative humidity control and adsorption of hydrocarbons.

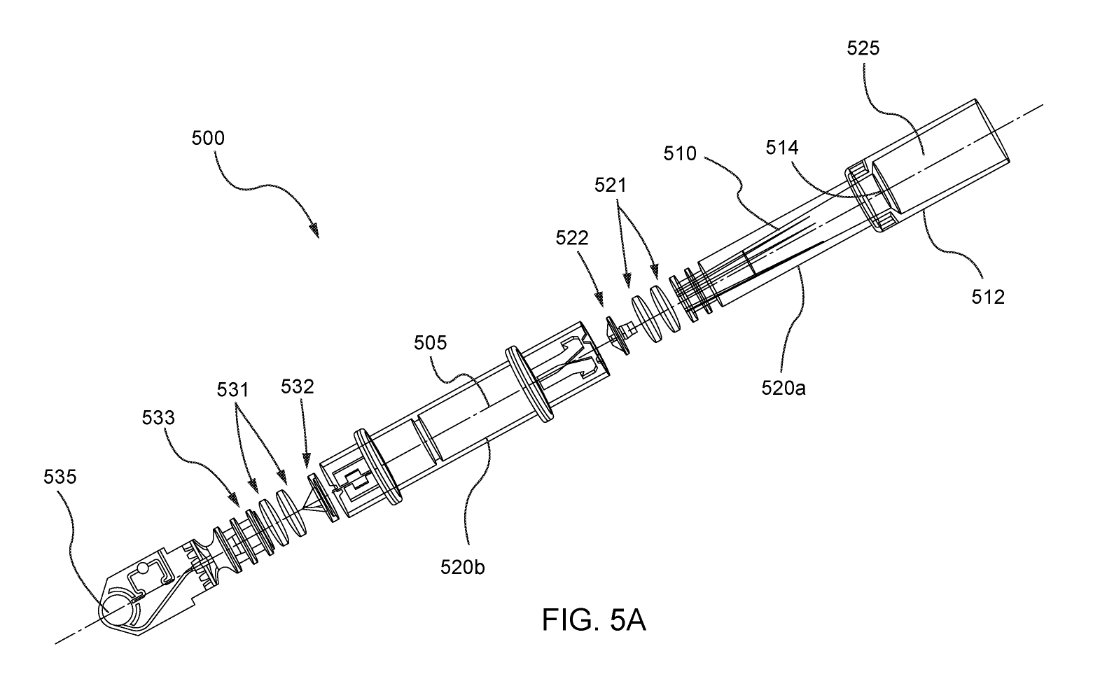

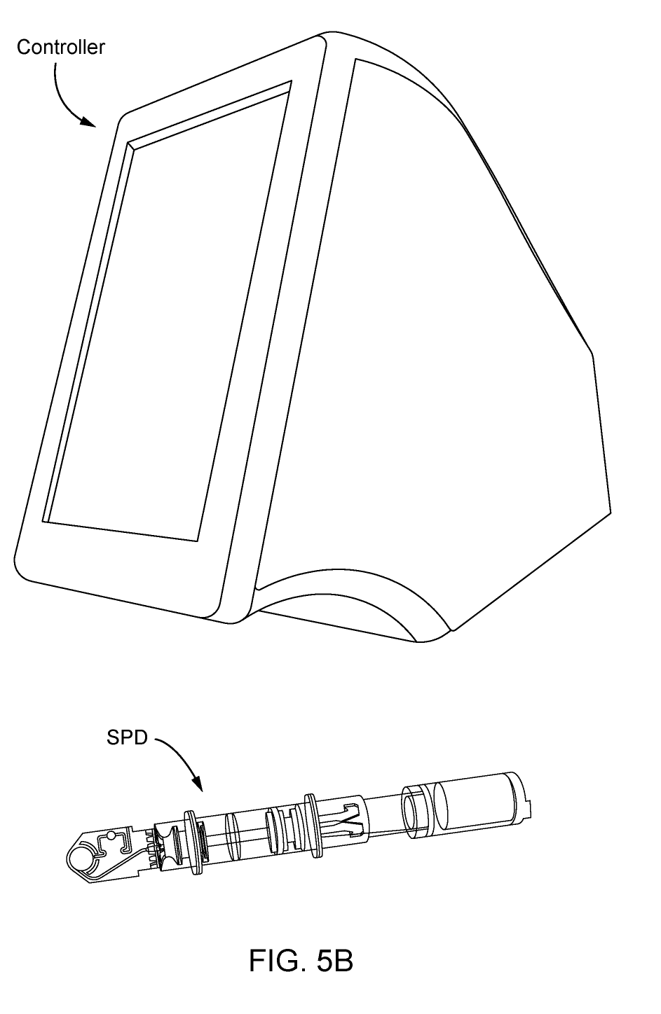

FIG. 5A shows a schematic of an SPD 500 using a spike to transport samples. The SPD 500 includes a reservoir 525 separated from a cap 512 via a breakable seal 514. The reservoir 525 can store chemistries for sample extraction. The reservoir 525 is attached to a swab (not shown in FIG. 5A) at location 510. The swab is insertable into a first compartment 520a. The first compartment 520a can be configured to receive the swab and form an air-tight seal with a module (e.g., a sample input module) that includes the swab.

The first compartment 520a is coupled to a second compartment 520b via two 0-rings 521 and a valve 522. In some embodiments, the valve 522 can be a one-way valve such that fluid can flow from the first compartment 520a to the second compartment 520b, but not the other way around. In some embodiments, the two compartments 520a and 520b can be translated with respect to each other so as to draw the sample from the first compartment 520a to the second compartment 520b.

The SPD 500 also includes a third compartment 535 for nucleic acid amplification and/or detection. The third compartment 535 is coupled to the second compartment 520b via two O-rings 531, a spike 532 (also referred to as a hole punch 532), and a membrane 533. The spike 532 can be moved along the longitudinal axis of the SPD 500. During use, a user can move the spike 532 to pierce the membrane 533 so as to transport the sample from the second compartment 520b to the third compartment 533 for amplification and/or detection. In some embodiments, a device (e.g., the device 200 shown in FIG. 2) can move the spike 532 into position. In some embodiments, the spike 532 can be fixed and the user or the device can move the third compartment 535 toward the spike 532 to pierce the membrane 533.

FIG. 5B shows a photo of an SPD that can be structurally and/or functionally similar to the SPD 500 shown in FIG. 5A. FIG. 5B also shows a controller to actuate the diagnostic device, such as the device 200 shown in FIG. 2 and described above. The controller includes a touch screen for a user to conveniently operate the diagnostic device for sample preparation and/or detection.

Methods of Inline Sample Preparation

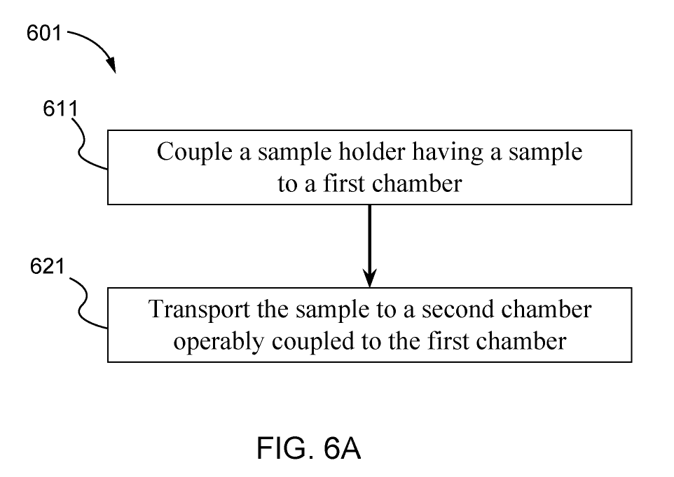

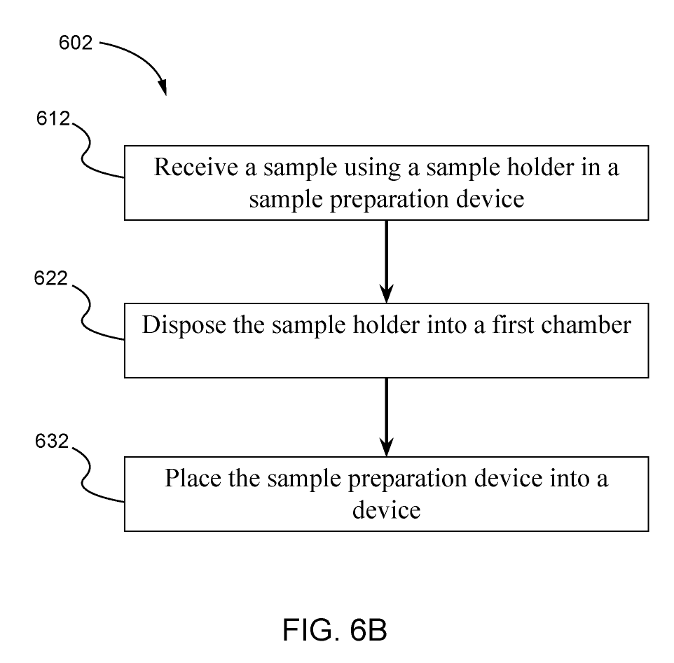

FIG. 6A illustrates a method 601 of inline sample preparation, according to some embodiments. The method 601 includes, at 611, coupling a sample holder (e.g., the sample holder 110 shown in FIG. 1) to a first chamber. The sample holder has a sample disposed therein. The sample holder and the first chamber have a common longitudinal axis when coupled. The first chamber includes at least one extraction reagent for extracting nucleic acid from the sample. The method 601 also includes transporting at least a portion of the sample to a second chamber operably coupled to the first chamber and disposed along the common longitudinal axis. The second chamber includes at least one reagent, such as an oligomer for example, for amplifying the nucleic acid.

In some embodiments, the sample holder can hermetically seal the first chamber and the second chamber so as to allow syringe operation and draw the sample through different portion in the first chamber and/or the second chamber.

In some embodiments, the method 601 further includes receiving the sample, which can include a sputum sample, a urine sample, a feces sample, a blood sample, and/or a buccal swab, among others.

In some embodiments, the method 601 further includes receiving the sample using a swab. In some embodiments, the method 601 further includes receiving the sample using a pipette. In some embodiments, the method 601 further includes receiving the sample using a syringe.

In some embodiments, the extraction reagent includes a liquid reagent stored in a first storage unit within the first chamber. In such embodiment, the method 601 can further include transporting the liquid reagent from the first storage unit to the sample holder so as to cause the liquid reagent to be in contact with the sample.

In some embodiments, the method 601 can extract more than one type of nucleic acid simultaneously. For example, the method 601 can include extracting first nucleic acid using a first extraction reagent preloaded in the first chamber and extracting second nucleic acid using a second extraction reagent preloaded in the first chamber. In this case, a single SPD as described above can be used and multiple reagents can be preloaded in the SPD.

In some embodiments, the method 601 further includes amplifying the first nucleic acid using a first oligomer preloaded in the second chamber and amplifying the second nucleic acid using a second oligomer preloaded in the second chamber.

In some embodiments, coupling the sample holder includes disposing the sample holder into a first compartment operably coupled to a second compartment. In this case, the method 601 can further include moving the second compartment away from the first compartment so as to draw at least a portion of the sample from the first compartment to the second compartment through a first valve disposed within the first compartment. In some embodiments, the first valve can be a one-way valve to prevent fluid flow from the second compartment back to the first compartment so as to reduce contamination.

In some embodiments, the extraction reagent can include a lyophilized reagent disposed on the first valve. In some embodiments, the extraction reagent includes a lyophilized reagent disposed on an inner wall of the first compartment and/or the inner wall of the second compartment. In some embodiments, the extraction reagent can be disposed on any filter included in the first compartment and/or the second compartment.

In some embodiments, the method 610 further includes placing the first chamber into a device including a motor to move the second compartment away from the first compartment. The device can be substantially similar to the device 200 shown in FIG. 2 and described above. In some embodiments, the device can have a manual mode, in which a user can manually move the compartments of the first chamber for sample transportation. In some embodiments, the device can have an automatic mode, in which a user can just push a button and the device can automatically move the compartments of the first chamber for sample transportation.

In some embodiments, the method 601 includes moving the second compartment closer to the first compartment so as to transport at least a portion of the sample through a second valve disposed within the second compartment and into the second chamber. The second chamber includes at least one oligomer, which can be disposed on the second valve or in the second chamber. The oligomer can be naturally occurring, or synthesized.

In some embodiments, the amplification of the nucleic acid can be achieved via an XCR′ process. In some embodiments, the amplification of the nucleic acid can be achieved via a PCR process. The amplified nucleic acid can then be detected by, for example, observing fluorescence of the sample in response to light illumination.

In some embodiments, the sample holder, the first chamber and the second chamber can be disposable. For example, after each amplification and detection, the SPD is discarded, instead of being reused.

FIG. 6B illustrates another method 602 of inline sample preparation. The method 602 includes, at 612, receiving a sample using a sample holder in a sample preparation device, which further includes a first chamber operably coupled to a second chamber. The method 620 also includes, at 622, disposing the sample holder into the first chamber and, at 632, placing the sample preparation device into another device/system. In response to an instruction from a user, the second device can be configured to extract nucleic acid from the sample using at least one extraction reagent preloaded in the first chamber, transport at least a portion of the sample to the second chamber, and amplify the nucleic acid using at least one oligomer preloaded within the second chamber.

In some embodiments, in response to the instruction from the user, the device is further configured to illuminate the second chamber and detect the nucleic acid via fluorescence. In some embodiments, the device can include a memory to store processor-executable instructions and a processor to execute the instructions so as to carry out the sample extraction, amplification, and detection. The user instruction in this case can be just a push of a start button.

In some embodiments, disposing the sample holder into the first chamber includes hermetically sealing the sample preparation device. This can facilitate the syringe movement of the first chamber when transporting the sample.

In some embodiments, the method 602 includes receiving at least one of a sputum sample, a urine sample, a feces sample, a blood sample, or a buccal swab, among others. The sample can be received by a swab, a pipette, and/or a syringe.

In some embodiments, the extraction reagent includes a liquid reagent stored in a first storage unit within the first chamber. In this case, in response to the instruction the device controls the sample preparation device to transport the liquid reagent from the first storage unit to the sample holder so as to cause the liquid reagent to be in contact with the sample. For example, the device can move the syringe structure of the sample preparation device to make the sample transportation.

In some embodiments, in response to the instruction the device further controls the sample preparation device to extract first nucleic acid using a first extraction reagent preloaded in the first chamber and extract second nucleic acid using a second extraction reagent preloaded in the first chamber. In some embodiments, the method 602 can include extracting nucleic acid from two, or more than two, types of target or microorganism simultaneously.

Described hereon with reference to extraction of nucleic acid from one target/microorganism, in some embodiments, in response to the instruction the device amplifies the first nucleic acid using a first oligomer preloaded in the second chamber and amplifies the second nucleic acid using a second oligomer preloaded in the second chamber.

In some embodiments, disposing the sample holder into the first chamber includes disposing the sample holder into a first compartment operably coupled to a second compartment. In response to the instruction the device moves the second compartment away from the first compartment so as to draw at least a portion of the sample from the first compartment to the second compartment through a first valve (or a first permeable membrane) disposed within the first compartment.

In some embodiments, in response to the instruction the device moves the second compartment closer to the first compartment so as to transport the portion of the sample through a second valve (or second permeable membrane) disposed within the second compartment and into the second chamber.

In some embodiments, in response to the instruction the device amplifies the nucleic acid via extreme chain reaction (XCR′). In some embodiments, in response to the instruction the device amplifies the nucleic acid via PCR. In some embodiments, in response to the instruction the device detects the nucleic acid using fluorescence.

In some embodiments, a temperature from about 38° C. to about 100° C. is used for the extraction process. In some embodiments, a temperature of about 95° C. is used for the extraction process. In some embodiments, a temperature of about 80° C. is used for the extraction process.

In some embodiments, the sample is treated at the elevated temperature for at least about 2 minutes, at least about 3 minutes, at least about 4 minutes, at least about 5 minutes, at least about 10 minutes, at least about 15 minutes, at least about 30 minutes, or at least about 60 minutes. In some embodiments, the sample is treated at the elevated temperature for at least about 5 minutes.

### Example 1—Inline Sample Preparation in Group A Strep Test

### Introduction

This example describes inline sample preparation in XCR™ GAS Direct Test (also referred to as Giza Group A Strep test, or simply Group A test in this application), which is a point of care in vitro diagnostic test for the qualitative detection of Streptococcus pyogenes (Group A β-hemolytic Streptococcus) in throat swab specimens. The instrumented test system uses the molecular technology of Xtreme Chain Reaction (XCR™), which is a variant of PCR. The test detects Streptococcus pyogenes by targeting a segment of the Streptococcus pyogenes genome.

XCR™ GAS Direct Test is intended to aid in the rapid diagnosis of Group A Streptococcus (GAS) bacterial infections in adults and children who present in a medical care facility with signs and symptoms of the pharyngitis.

Acute pharyngitis is a common presenting symptom, accounting for an estimated 15 million patient visits in 2006. Among pharyngitis cases, Group A streptococcus (GAS; Streptococcus pyogenes) causes 5 to 15% of cases in adults and 20 to 30% of cases in children. Streptococcal pharyngitis occurs most frequently among children 5-15 years of age, resulting in an estimated economic burden in the United States of $224 million to $539 million per year with a large portion of the associated costs attributable to parents' lost time from work. The cost per case is estimated at $205. In children with sore throat, pharyngitis is frequently treated empirically with anti-microbials resulting in overtreatment. While symptom scoring systems improve diagnosis, even with a high clinical suspicion of Streptococcal pharyngitis, there remains significant overtreatment of streptococcal pharyngitis.

At clinical presentation, point of care testing today is typically performed using antibody-based lateral flow detection tests. These are rapid and completed within 5 minutes. However, test sensitivities are comparatively low, demanding follow up culture for negative results. This problem may be addressed through sensitive DNA amplification tests. However, in at least one case, a 501(k)-cleared and CLIA-waived molecular diagnostic test (Alere i) still requires follow up culture for negative results and is therefore not a significant improvement over the less expensive antibody-based test. In another cases, sophisticated disposable components lead to high cost per test (e.g., LIAT™). There remains a need for rapid, sensitive, specific, and affordable GAS testing at the point of care.

XCR™ techniques can allow sub 10-minute DNA/RNA amplification with the sensitivity and multiplex capabilities of PCR. Combined with a simple “filter/heat/go” sample preparation described above, XCR™, rapid amplification provides the necessary turnaround time and low cost to fill the unmet need in near-patient GAS testing. Patients can be treated appropriately, thereby lowering the economic burden associated with Streptococcal pharyngitis, and decreasing inappropriate antibiotic therapy.

The Platform

The platform described herein includes a sample preparation device (SPD) integrated with an electro-optical instrument. The platform can perform rapid, simplified, and automated molecular diagnostic testing. The platform has three components: (1) nucleic acid amplification chemistry called XCR™; (2) a disposable SPD; and (3) an instrument that performs DNA extraction, amplification, and detection of DNA.

The nucleic acid amplification chemistry, XCR™, uses the identical biochemical reagents, primers and probes as are used in PCR, but amplifies more rapidly by compressing thermal cycling temperature ranges. XCR™ retains the single-copy sensitivity of PCR while providing resistance to inhibitors and rapid amplification (5-10 minutes to completion). Assays are designed using PCR design software.

The SPD, which can be substantially similar to the apparatus shown in FIGS. 1, 3, and 5A, here contains a syringe-like system for moving fluids unidirectionally through filtration, heating, and finally to an amplification chamber that houses lyophilized amplification reagents and provides for optical detection of fluorescence. After throat swabbing, replacement of the cap produces a closed system to contain patient biological material and amplified nucleic acids.

The instrument, which can be substantially similar to the device 200 shown in FIG. 2, performs amplification and detection. Detection is achieved by standard chemistries using fluorescence detection of hydrolysis probes. Total testing time, from sample to results, can be less than 15 minutes. The test can have a built-in Sample Process Control (SPC) to assure the test performs correctly. A processor (e.g., included in a computer) embedded within the instrument can perform the following: manages the electroptics and the minimal mechanical movements, compiles test data and results, and uploads the data to the touch screen as well as to servers using wireless telemetry.

Test Principles and Workflow

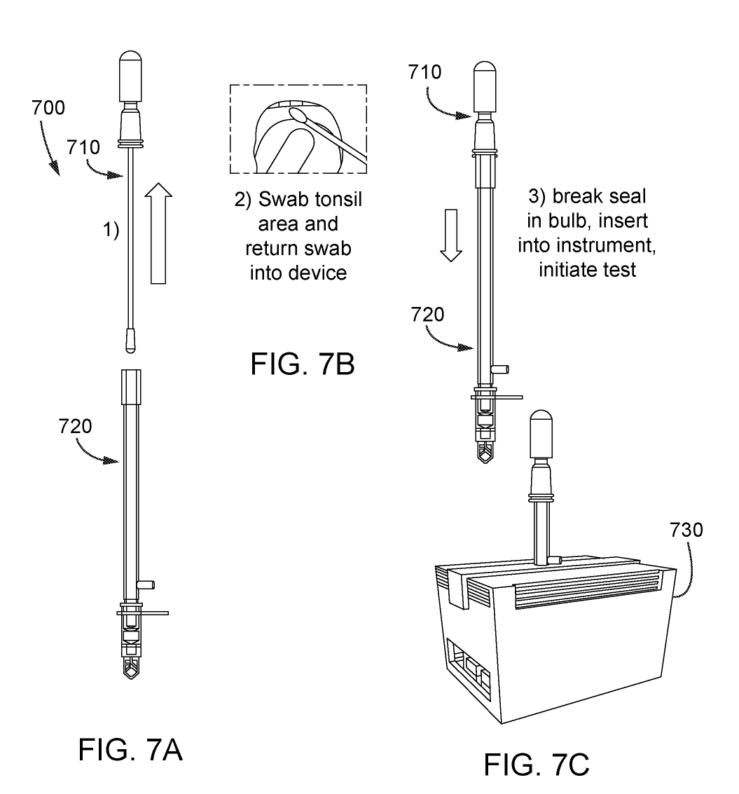

FIGS. 7A-7C illustrate a workflow 700 of Strep A Test, which uses nucleic acid test chemistry XCR™ and an assay protocol for bacterial DNA detection. First, the SPD is uncapped, a sterile swab 710 is removed from a processing chamber 720 in the SPD, as shown in FIG. 7A. FIG. 7B shows that the sterile swab 710 is used to take a throat sample in the tonsillar area. The swab is then snapped back into the SPD and the operator then breaks a seal in the bulb to release lysis buffer onto the swab, as shown in FIG. 7C.

Next, the SPD is inserted into the instrument 730 and the operator initiates the test using a touch screen interface, also shown in FIG. 7C. Within the instrument, the lysis buffer briefly bathes the swab and the product is drawn across the swab and through two filters into a second chamber. This is accomplished using a modified syringe and a stepper motor within the instrument. During filtration, a Sample Process Control bacterium such as S. pasturianus, for example, can be automatically introduced into the sample. After filtration, bacteria are lysed by heating and the sample is pushed, without dilution, into the amplification chamber, simultaneously rehydrating and mixing the lyophilized PCR reagents.

The Strep A primer and probe set is designed for the detection of a conserved coding sequence that is unique to Strep A bacteria. Thermal cycling takes place, allowing primer annealing, extension, and amplicon denaturation, exactly as for PCR. Signal is generated by hydrolysis of fluorogenic probes specific to the target DNA. Fluorescence intensities are continuously monitored, and the instrument's included software automatically analyzes the fluorescence intensities to determine positive or negative results.

In the work flow 700, no reagent preparation or additional steps are needed. Because all the reagents are contained within the Sample Prep Device and no sample or reagent are removed from the tube, cross-contamination between samples is minimized.

XCR™ Chemistry

XCR™, or Xtreme Chain Reaction, uses standard PCR reagents and enzymes, with two differentiating features: it is 5×-10× faster than PCR, while maintaining PCR-like sensitivity, and the method has innate resistance to inhibition, simplifying sample preparation.

Sample Preparation Device

As shown in FIGS. 7A-7C, the SPD includes a dry swab 710 integrated with a bulb. The swab is removed using the bulb as a handle, the patient throat is swabbed, and the swab is snapped back into the SPD, forming a sealed, enclosed container. The cell lysis buffer contained within the bulb is released by breaking a seal, and the SPD is inserted in to the instrument 730. The operator then initiates the test using a simple user interface on the instrument.

The swab is washed with cell lysis buffer in the upper portion and the instrument then draws this buffer through filters, removing insoluble material. The buffer is then heated, and the instrument pushes the buffer into the amplification chamber, rehydrating and mixing lyophilized amplification reagents. Inclusion of a sample process control organism, the food bacterium Lactococcus lactis, serves to verify correct sample extraction, amplification and detection.

The instrument uses simple hardware to achieve amplification and detection, with sophisticated software to report results and to integrate with laboratory and/or office information systems. The instrument has a simple stepper motor to produce two fluid movement steps within the SPD, a heater to boil the sample and lyse cells, a second pair of heaters to achieve thermal cycling, and an optical detection block to interrogate fluorescence generated during the amplification phase.

After insertion of the SPD module, the user operates a touch screen and barcode scanner to identify the sample and initiate the test. Initially, a motor within the instrument engages with the SPD and draws a modified syringe upwards, creating a vacuum that pulls the cell lysis buffer across the swab and through a set of filters that remove insoluble material. Heaters then boil the sample. The motor then reverses, breaking a seal, and pushes the sample through a lyophilized reagent cake into the amplification chamber. During the reversal from pulling the syringe to pushing, fluid backflow is prevented using a one-way valve. As the amplification chamber is filled, reagents are mixed using fluidic pathways, and the exit port is automatically sealed in the presence of liquid, creating a closed system. Heater blocks then produce thermal cycling, and optical windows adjacent to the amplification chamber enable fluorescence detection by the optical block. A post-test UV exposure of the instrument sample serves as a precautionary prevention of contamination.

The electromechanical systems can use commercial off-the-shelf components where possible. However, the novel optical block can use a camera to detect fluorescence with novel features designed to maximize fluorescence sensitivity. For example, the platform can have built-in capability to achieve high multiplex amplification and detection.

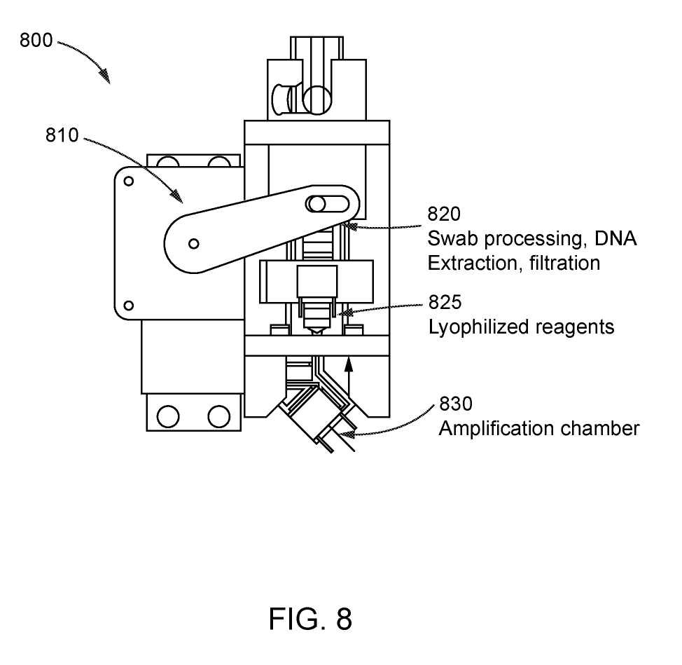

FIG. 8 illustrates a diagnostic instrument 800 and automated swab processing. This instrument 800 is also referred to as a portable Nucleic Acid Test System (pNATS) or an XCR′ portable diagnostic system. The instrument includes a motor 810 to produce two fluid movement steps described above.

The fluid movement steps can process the swab, including DNA extraction and filtration, in a processing chamber 820 of the SPD. Lyophilized reagents 825 can be included in the SPD as SPC or for amplification. The extracted DNA at 820 is then transported to an amplification chamber 830 for amplification and/or detection.

In some embodiments, the motor 810 drives a modified syringe that pulls lysis buffer across the swab, washing it and filtering out insoluble material. After boiling, the motor 810 reverses and pushes the sample through a filter a passage containing the lyophilized amplification reagents 825, filling the amplification chamber 830. For simplicity, heating elements for amplification and the optical block are not shown in FIG. 8.

Controls

The Strep A Assay can have 3 controls: (1) sample process control, (2) positive control and (3) negative control.

The sample process control (SPC) comprises a lyophilized, food grade, gram positive bacterium that is present in the SPD device. The lactococcus, bearing a plasmid that contains the target sequence, is automatically introduced into the cell lysis buffer during the filtration step, and its target DNA is detected in a separate channel by SPC specific primers and probe to avoid false negative test results due to excessive sample inhibition. If the target and SPC cycle threshold values (Ct) and fluorescence endpoints are not within an expected range and Strep A is not detected, the assay run report indicates “Invalid Assay, Repeat test.”

The positive control is provided in the Strep A Assay QC Kit. The positive control will comprise inactivated Strep A bacteria at approximately 10³ bacteria per swab.

The negative control is provided in the Strep A Assay QC Kit, comprising a swab containing 10⁵ cells Streptococcus salivarius, a commensal apathogenic species that does not react in the Strep A test.

Extreme Chain Reaction (XCR™)

This section relates to DNA amplification hereinafter referred to as “Dynamic Flux Amplification” or “DFA.” In some embodiments, the term(s) “XCR™”, “extreme chain reaction”, and/or variants thereof, can be interchangeably used with the terms “DFA”, “dynamic flux amplification”, and/or variants thereof.

Generally, DFA refers to specific techniques of DNA and RNA amplification. DFA takes advantage of the fact that DNA amplification can take place within a fairly narrow temperature range. Once the melting temperature (Tm) of the sequence of interest is determined, the DNA sample may be heated to that temperature or 1 to 5 degrees C. above that temperature. This defines the upper parameter of the heating and cooling cycle. The Tm of either the primers or the probes, (whichever possesses the lower Tm) defines the lower parameter of the heating and cooling cycle, within 1 to 5 degrees C.

In practicing DFA, it is generally beneficial to use primers with a Tm as close as possible to the Tm of the sequence of interest so that the temperature may be cycled within a narrow range. The result of this narrow cycling is a dynamic opening and closing of a duplex between complementary nucleic acids comprising the sequence of interest as opposed to the complete, or nearly complete denaturing of the entire DNA strand.

The present existing primers (e.g., primers that are tested) can target nucleic acid product that contains fewer nonspecific products. Thus, the amplified target nucleic acids products can be overall more specific and sensitive for quantitative PCR and genotyping target detection applications as described herein.

“Rational design” of oligonucleotide primers can include the selection via calculation, experiment, or computation of primers that have the desired melting temperature (Tm). The rational design can include selection of a specific primer sequences with the appropriate CG to obtain the desired Tm. Also, the rational design can include modifications to the primers that include internucleotide modifications, base modifications, and nucleotide modifications.

DFA Primer Design Methodology

In some embodiments, methods are provided for selecting primers for DFA that flank a variable sequence element of interest on a target nucleic acid.

In some embodiments, primers are selected to have a Tm with the target nucleic acid (primer:target Tm) that is within a narrow range of the Tm of the target nucleic acid (target:target Tm). The specific, narrow temperature range used for such an amplification of the target nucleic acids can be dependent on the melting profile of the target nucleic acid, and thereby the sequence of the target nucleic acid being amplified. As such, the narrow temperature range can be used as a target temperature range in order to identify and/or generate specific primers that have sufficiently high Tm values when hybridized with the target nucleic acid.

DFA Primer Design—Overlapping Annealing/Denaturing Curves

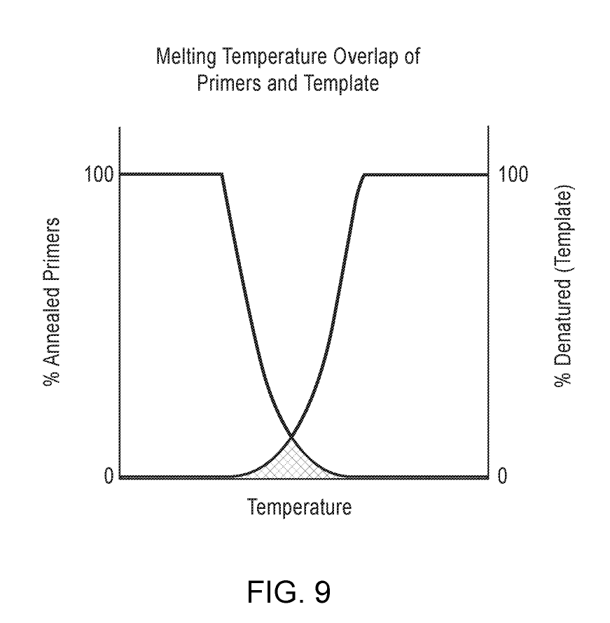

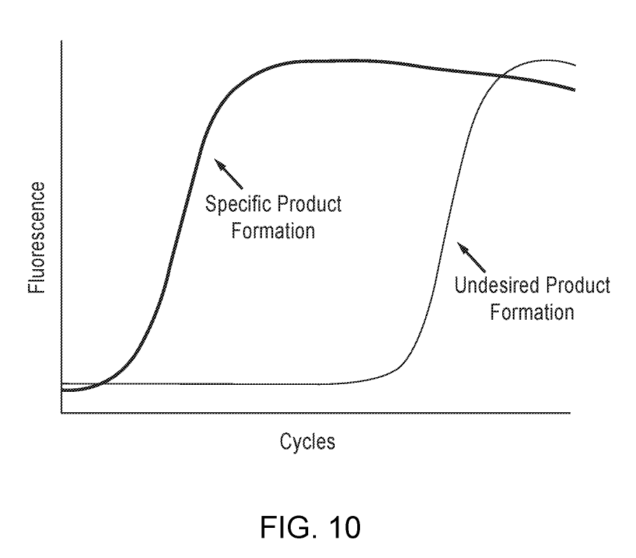

Accordingly, the Tm values of the primers can be overlapping within the temperature range of annealing and/or denaturing of the target nucleic acid (See, FIG. 9). FIG. 9 can be contrasted with FIG. 10 to illustrate the design of the primers to have the Tm within a range of the Tm of the target nucleic acid. FIG. 10 shows that conventional amplification with primers and a target nucleic acid are devoid of having a temperature overlap (as shown in FIG. 9) and require extreme temperature variations during amplification, corresponding to denaturation, annealing and extension cycles, to produce an amplified product. Such extreme temperature ranges allow for the formation of undesired products.

DFA Primer Design—Iterative Design

In some embodiments, an iterative design process can be employed to select and/or optimize primers for specific target nucleic acid sequences to be amplified and/or detected. Advantageously, the iterative method allows the formation of a specific target nucleic acid by using a narrow range of thermal conditions where both the target nucleic acid and the oligonucleotide primers hybridized with the target nucleic acid are in a dynamic flux of annealing and denaturing. Such a dynamic flux of annealing and denaturing can result in a specific amplification of the target nucleic acid with a commensurate decrease in the formation of nonspecific amplification products. The implications of such iterative methods for selecting and/or optimizing primers provides for the use of low cost dyes in lieu of more expensive custom oligonucleotide probes, such as those having fluorescent labels, can allow for quantitative PCR or high resolution denaturation to be used in analyzing the sequence of the target nucleic acid. Also, the iterative method can provide primers that function in the absence of exquisite thermally controlled instruments for the formation of amplification products.

That is, the primers can operate within a narrow temperature range in order to amplify the target nucleic acid, allowing nucleic acid amplification to be used in a much broader range of uses. A number of methods have been described in the art for calculating the theoretical Tm of DNA of known sequence, including, e.g., methods described by Rychlik and Rhoads, Nucleic Acids Res. 17:8543-8551 (1989); Sambrook, J. et al., Molecular Cloning: A Laboratory Manual, Cold Spring Harbor Laboratory Press, Cold Spring Harbor, N.Y. (1989); and Breslauer et al., Proc Natl Acad. Sci. 83: 3746-3750 (1986).

Such an iterative process can include identifying an initial target nucleic acid sequence as the target amplicon, wherein the target nucleic acid sequence can be associated with a particular biological activity, such as possible drug resistance. The target nucleic acid sequence is then amplified in order to produce an amplified product, and the Tm value of the amplified product (e.g., amplicon) is determined using conventional melting curve analysis. The melting curve analysis is then utilized to determine or compute new primers or primer sets for use in the amplification of the target nucleic acid.

The determined or computed primers are then designed with primer Tm values within the range of the melting peak generated by the melt of the amplified product. The primers are then prepared or synthesized to have the designed primer Tm values.

DFA Primer Design—Oligonucleotide Chemical Modification

In some embodiments, primers can be configured to have a Tm that is within a narrow range of the Tm of the target nucleic acid by chemically modifying the oligonucleotides. Oligonucleotide synthesis chemistries may be used to increase the Tm values of the primers so they correspond to the temperature range of the Tm of the target nucleic acid. Such chemistries may use modified bases (e.g., Super G, A, T, C), LNA, or PNA, or other such oligonucleotide stabilizing chemistries. Also, additional oligonucleotide hybridization stabilizing chemistries may be developed that can be used for this application.

For example, primers synthesized with both phosphodiester linkage chemistry, and LNA chemistries can be used to provide primer Tm values close to the Tm values of the target nucleic acid sequence. However, it is possible that certain target nucleic acids may have Tm values lower than that of the primers, and a hybridization destabilizing chemistry may be helpful to decrease the primer Tm values so that the primer Tm value is within a range of the Tm values of the target nucleic acid sequence.

DFA Primer Design—Melting Curve Analysis

In some embodiments, methods are provided for refining the design of the primers to minimize the temperature range for the specific amplification of the target nucleic acid sequence. As such, the target nucleic acid is amplified with standard reaction thermal cycling conditions to ensure the target nucleic acid sequence is amplified. The amplification can be monitored using real-time PCR with a double-stranded DNA binding dye, such as SYBR, LCGreen, LCGreen+, Eva dye, or the like.

The amplified target nucleic acid can be subjected to a melting curve analysis to determine the actual Tm value of the target nucleic acid sequence. The melting peak, which can be expressed as −dF/dT, is generated from melting the amplified target nucleic acid and can have a range similar to a distribution curve across a defined temperature range. At the low temperature end, the amplified target nucleic acid template is partially denatured. At the uppermost temperature the entire sample of amplified target nucleic acid is denatured. The temperature to denature the target nucleic acid during the amplification procedure is within this temperature distribution.

Initially, the uppermost temperature is recommended to ensure more complete denaturation. Subsequently, the lowermost temperature of the distribution curve can be used as the initial Tm for a set of designed primers for use in amplification before any iterative changes are made to the primers.

Confirmation of the narrow temperature range that the initial primers may be used with can be performed either in serial or in parallel experiments of ever increasing annealing temperatures.

Alternatively, the individual primers can be added to the amplified template and an additional melting curve analysis can be performed on the combined primer and template melting curves.

In any event, the Tm of the primers can be configured to overlap with a narrow temperature range that contains the Tm of the target nucleic acid sequence. The highest annealing temperature from these experiments where the target nucleic acid sequence is amplified specifically and efficiently can be considered as the temperature, which defines the optimal annealing temperature for the existing primers (e.g. primers that were tested). These same primers or slightly modified primers can then be resynthesized with additional hybridization stabilizing chemistries.

Modifications to the primers can change the Tm in the desired direction so that the primer Tm overlaps with a narrow temperature range that contains the Tm of the target nucleic acid sequence. This can be accomplished using online design tools, such as the LNA design tool available from Integrated DNA Technologies. Such design tools can be used to estimate the number of necessary LNA modifications to raise the Tm of the primer to better overlap with the melting curve of the target nucleic acid sequence.

In the instance the primer Tm values are greater than the highest melting temperature of the target nucleic acid sequence, it may be beneficial to redesign the primers to have a lower Tm. Alternatively, the quantity of divalent and/or monovalent cation salts or other destablizing reagents (e.g., AgCI, DMSO, etc.) that are used in the amplification protocol (e.g., PCR) may be reduced to destabilize the hybridization of these oligonucleotides to the template. In any event, a reduction in the primer Tm may be helpful in some instances.

DFA Primer Design—GC Content Modification

In some embodiments, the primer Tm can be modified by altering the GC content of the primer sequence. By changing the GC content, the primer Tm can be selectively changed. Usually, increasing the GC content can increase the Tm, and decreasing the GC content can decrease the Tm. However, there are instances that a high GC content is desired that will overly increase the Tm. In such instances, destabilizers can be used to enable the inclusion of high GC content primers or for the use of high GC content target nucleic acid sequences. The de-stabilizers can selectively decrease the temperature of the amplification procedure. Examples of destabilizers include DMSO, AgCI, and others.

DFA Thermal Cycling Ranges

In some embodiments, the primers can be prepared so that the target nucleic acid amplification or enrichment protocols can be performed at minimized temperature differences during the thermal cycling. This allows the thermal cycling to be done within a narrow temperature range so as to promote the formation of a specific product.

One range of thermal cycling can be within about 15° C. of the target nucleic acid. Tm, or within 10° C. of the target nucleic acid Tm, or within 5° C. of the target nucleic acid Tm, or within 2.5° C. of the target nucleic acid Tm, or within 1° C. of the target nucleic acid Tm or even substantially the same Tm as that of the target nucleic acid Tm.

In some embodiments, the thermal cycling conditions for the amplification of the target nucleic acid spans the range of the Tm peak +/− about 1° C. to 15° C. of the target nucleic acid sequence

In some embodiments, the thermal cycling conditions for the amplification of the target nucleic acid spans the range of the Tm peak +/− about 1° C. to 10° C. of the target nucleic acid sequence.

Or, in some embodiments, the thermal cycling conditions for the amplification of the target nucleic acid spans the range of the Tm peak +/− about 1° C. to 5° C. of the target nucleic acid sequence.

In some embodiments, the thermal cycling conditions for the amplification of the target nucleic acid spans the range of the Tm peak +/− about 5° C. to 15° C. of the target nucleic acid sequence.

In some embodiments, the thermal cycling conditions for the amplification of the target nucleic acid span the range of the Tm peak +/− about 5° C. to 10° C. of the target nucleic acid sequence.

In some embodiments, the thermal cycling conditions for the amplification of the target nucleic acid span the range of the Tm peak +/− about 5° C. of the target nucleic acid sequence.

In some embodiments, the thermal cycling conditions for the amplification of the target nucleic acid span the range of the Tm peak +/− about 2.5° C. of the target nucleic acid sequence.

Such narrow temperature ranges make it possible to amplify specific target nucleic acids without thermal cycling between temperatures corresponding to the normal stages of PCR amplification (denaturation, annealing, and extension).

Also, it is possible to perform amplifications and enrichments in commercial temperature-controlled instruments that can be set at selected temperatures or be varied within narrow temperature ranges, such as an oven, heating block, or the like.

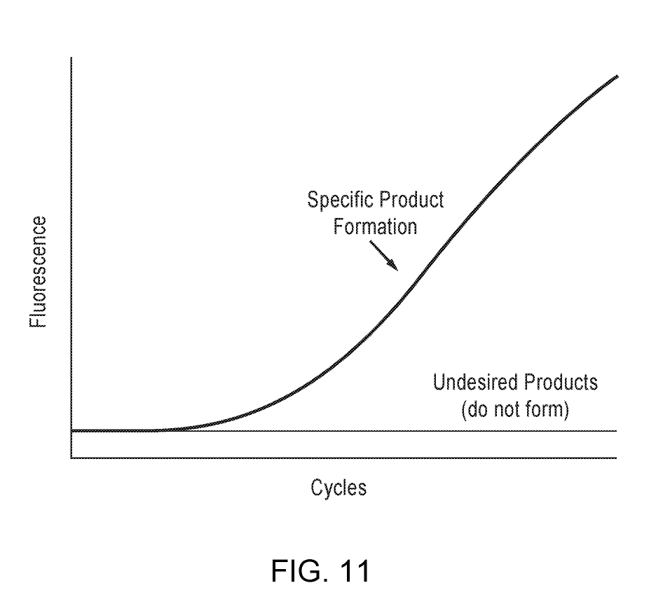

FIG. 11 illustrates the graph of a narrow temperature range PCR amplification with the same target nucleic acid sequence as shown in FIG. 10, which shows more specific product formation and less undesired products are formed.

In some embodiments, the temperatures of the thermos-cycling can be selected in a narrow temperature range to substantially limit amplification to amplifying the target nucleic acid sequence. As such, the thermal cycling conditions can be modified to amplify the target nucleic acid sequence by modifying the annealing temperature to be substantially the same as the lower temperature base of the melting peak for the amplicon. Also, the thermal cycling conditions can be modified to amplify the target nucleic acid sequence by modifying the annealing temperature to be substantially the same as the higher temperature base for the melting peak of the amplicon.

In some embodiments, the primer Tm can be selected so that the amplification of the target nucleic acid can be performed at a temperature that ranges between about 75° C. to about 90° C. Such a temperature range, or narrowed 5° C. to 10° C. range therein, can be used for the amplification of DNA and/or RNA target nucleic acid sequences to reduce the formation of non-specific products during the amplification (e.g., PCR) process. In some aspects, the uppermost temperature range during the amplification reaction is 100° C., or 99° C., or 98° C., or 97° C., or 96° C., or 95° C., or 94° C., or 93° C., or 92° C., or 91° C., or 90° C. In some embodiments, the uppermost temperature range during the amplification reaction is less than about 100° C., or 99° C., or 98° C., or 97° C., or 96° C., or 95° C., or 94° C., or 93° C., or 92° C., or 91° C., or 90° C. In some embodiments, the uppermost temperature range during the amplification reaction is less than about 90° C.±10° C. In some embodiments, the uppermost temperature range during the amplification reaction is less than about 95° C.±10° C. In some embodiments, the uppermost temperature range during the amplification reaction is less than about 95° C.±5° C.

In some embodiments, the primer Tm can be selected so that the amplification is performed at isothermal amplification conditions in the Tm range of the target nucleic acid sequence to ensure appropriate product formation.

In some embodiments, the present disclosure includes a method of designing a primer set having a Tm with a target nucleic acid that is within a narrow range from the Tm of the target nucleic acid sequence. As such, the primer set can be designed so that the primer Tm overlaps the distribution curve of the Tm of the target nucleic acid sequence. For example, the primer set can be used in real-time PCR assays so that the primer Tm overlaps the distribution curve of the Tm for the target nucleic acid sequence so that a narrow temperature range can be used to amplify the target nucleic acid sequence.

DFA pH Modification

In some embodiments, the conditions of the protocol for amplifying the target nucleic acid sequence can be modified to an appropriate pH to increase the specificity of selectively amplifying the target nucleic acid over other nucleic acids. As such, the use of an appropriate pH can increase the ability to selectively amplify the target nucleic acid sequence. This can include the use of an amplification buffer that can enable the activation of chemically inactivated thermal stable DNA polymerases. Also, adjusting the pH with selected amplification buffers can allow for the amplification protocol to be performed at reduced temperatures, such as those temperatures ranges that have been recited herein.

In some embodiments, the pH of the amplification buffer can be adjusted so as to allow for the conversion of a chemically inactivated enzyme to the activated state. As such, an enzyme may be activated in a slightly acidic condition; however, basic pH values may be used for some enzymes. For acid-activated enzymes, standard Tris-based PCR buffers can have significant temperature dependence (e.g., reducing by 0.028 pH units per degree C.). Complete activation of the enzyme (e.g., chemically inactivated thermal stable DNA polymerase) from the inactivated state can require the pH to be less than about 7, more preferably less than about 6.75, and most preferably less than 6.5.

In some embodiments, the amplification protocol includes the use of lower pH buffers so that the amplification can be performed at lower activation temperatures. For example, for every 10° C. below 95° C., the enzyme activation temperature can be lowered by 0.3 pH units. However, limits to this approach are entirely a function of the dye chemistry used for the real-time detection of the amplified template (e.g., Fluorescein-based detection has significantly reduced fluorescence below pH 7.3).

DFA Modulation of Amplicon Size

In some embodiments, the design of the primers and/or amplification conditions can be modulated so as to modulate the size of the target nucleic acid sequence being amplified. This can include modulating the design of the primers and/or amplification conditions so that the size of the amplicon is significantly larger than that of the combined primers only. This can include the amplicon being 1-3 nucleotides longer than the primers, or 2 times larger than the primers, or 5 times larger than the primers, and more preferably 10 times larger than the primers.

### CONCLUSION

While only a few embodiments of the present disclosure have been shown and described, it will be obvious to those skilled in the art that many changes and modifications may be made thereunto without departing from the spirit and scope of the present disclosure as described in the following claims. All patent applications and patents, both foreign and domestic, and all other publications referenced herein are incorporated herein in their entireties to the full extent permitted by law.

The methods and systems described herein may be deployed in part or in whole through a machine that executes computer software, program codes, and/or instructions on a processor. The present disclosure may be implemented as a method on the machine, as a system or apparatus as part of or in relation to the machine, or as a computer program product embodied in a computer readable medium executing on one or more of the machines. In embodiments, the processor may be part of a server, cloud server, client, network infrastructure, mobile computing platform, stationary computing platform, or other computing platform. A processor may be any kind of computational or processing device capable of executing program instructions, codes, binary instructions and the like. The processor may be or may include a signal processor, digital processor, embedded processor, microprocessor or any variant such as a co-processor (math co-processor, graphic co-processor, communication co-processor and the like) and the like that may directly or indirectly facilitate execution of program code or program instructions stored thereon. In addition, the processor may enable execution of multiple programs, threads, and codes. The threads may be executed simultaneously to enhance the performance of the processor and to facilitate simultaneous operations of the application. By way of implementation, methods, program codes, program instructions and the like described herein may be implemented in one or more thread. The thread may spawn other threads that may have assigned priorities associated with them; the processor may execute these threads based on priority or any other order based on instructions provided in the program code. The processor, or any machine utilizing one, may include memory that stores methods, codes, instructions and programs as described herein and elsewhere. The processor may access a storage medium through an interface that may store methods, codes, and instructions as described herein and elsewhere. The storage medium associated with the processor for storing methods, programs, codes, program instructions or other type of instructions capable of being executed by the computing or processing device may include but may not be limited to one or more of a CD-ROM, DVD, memory, hard disk, flash drive, RAM, ROM, cache and the like.

A processor may include one or more cores that may enhance speed and performance of a multiprocessor. In embodiments, the process may be a dual core processor, quad core processors, other chip-level multiprocessor and the like that combine two or more independent cores (called a die).

The methods and systems described herein may be deployed in part or in whole through a machine that executes computer software on a server, client, firewall, gateway, hub, router, or other such computer and/or networking hardware. The software program may be associated with a server that may include a file server, print server, domain server, internet server, intranet server, cloud server, and other variants such as secondary server, host server, distributed server and the like. The server may include one or more of memories, processors, computer readable media, storage media, ports (physical and virtual), communication devices, and interfaces capable of accessing other servers, clients, machines, and devices through a wired or a wireless medium, and the like. The methods, programs, or codes as described herein and elsewhere may be executed by the server. In addition, other devices required for execution of methods as described in this application may be considered as a part of the infrastructure associated with the server.

The server may provide an interface to other devices including, without limitation, clients, other servers, printers, database servers, print servers, file servers, communication servers, distributed servers, social networks, and the like. Additionally, this coupling and/or connection may facilitate remote execution of program across the network. The networking of some or all of these devices may facilitate parallel processing of a program or method at one or more location without deviating from the scope of the disclosure. In addition, any of the devices attached to the server through an interface may include at least one storage medium capable of storing methods, programs, code and/or instructions. A central repository may provide program instructions to be executed on different devices. In this implementation, the remote repository may act as a storage medium for program code, instructions, and programs.

The software program may be associated with a client that may include a file client, print client, domain client, internet client, intranet client and other variants such as secondary client, host client, distributed client and the like. The client may include one or more of memories, processors, computer readable media, storage media, ports (physical and virtual), communication devices, and interfaces capable of accessing other clients, servers, machines, and devices through a wired or a wireless medium, and the like. The methods, programs, or codes as described herein and elsewhere may be executed by the client. In addition, other devices required for execution of methods as described in this application may be considered as a part of the infrastructure associated with the client.

The client may provide an interface to other devices including, without limitation, servers, other clients, printers, database servers, print servers, file servers, communication servers, distributed servers and the like. Additionally, this coupling and/or connection may facilitate remote execution of program across the network. The networking of some or all of these devices may facilitate parallel processing of a program or method at one or more location without deviating from the scope of the disclosure. In addition, any of the devices attached to the client through an interface may include at least one storage medium capable of storing methods, programs, applications, code and/or instructions. A central repository may provide program instructions to be executed on different devices. In this implementation, the remote repository may act as a storage medium for program code, instructions, and programs.

The methods and systems described herein may be deployed in part or in whole through network infrastructures. The network infrastructure may include elements such as computing devices, servers, routers, hubs, firewalls, clients, personal computers, communication devices, routing devices and other active and passive devices, modules and/or components as known in the art. The computing and/or non-computing device(s) associated with the network infrastructure may include, apart from other components, a storage medium such as flash memory, buffer, stack, RAM, ROM and the like. The processes, methods, program codes, instructions described herein and elsewhere may be executed by one or more of the network infrastructural elements. The methods and systems described herein may be adapted for use with any kind of private, community, or hybrid cloud computing network or cloud computing environment, including those which involve features of software as a service (SaaS), platform as a service (PaaS), and/or infrastructure as a service (IaaS).

The methods, program codes, and instructions described herein and elsewhere may be implemented on a cellular network having multiple cells. The cellular network may either be frequency division multiple access (FDMA) network or code division multiple access (CDMA) network. The cellular network may include mobile devices, cell sites, base stations, repeaters, antennas, towers, and the like. The cell network may be a GSM, GPRS, 3G, EVDO, mesh, or other networks types.

The methods, program codes, and instructions described herein and elsewhere may be implemented on or through mobile devices. The mobile devices may include navigation devices, cell phones, mobile phones, mobile personal digital assistants, laptops, palmtops, netbooks, pagers, electronic books readers, music players and the like. These devices may include, apart from other components, a storage medium such as a flash memory, buffer, RAM, ROM and one or more computing devices. The computing devices associated with mobile devices may be enabled to execute program codes, methods, and instructions stored thereon. Alternatively, the mobile devices may be configured to execute instructions in collaboration with other devices. The mobile devices may communicate with base stations interfaced with servers and configured to execute program codes. The mobile devices may communicate on a peer-to-peer network, mesh network, or other communications network. The program code may be stored on the storage medium associated with the server and executed by a computing device embedded within the server. The base station may include a computing device and a storage medium. The storage device may store program codes and instructions executed by the computing devices associated with the base station.

The computer software, program codes, and/or instructions may be stored and/or accessed on machine readable media that may include: computer components, devices, and recording media that retain digital data used for computing for some interval of time; semiconductor storage known as random access memory (RAM); mass storage typically for more permanent storage, such as optical discs, forms of magnetic storage like hard disks, tapes, drums, cards and other types; processor registers, cache memory, volatile memory, non-volatile memory; optical storage such as CD, DVD; removable media such as flash memory (e.g. USB sticks or keys), floppy disks, magnetic tape, paper tape, punch cards, standalone RAM disks, Zip drives, removable mass storage, off-line, and the like; other computer memory such as dynamic memory, static memory, read/write storage, mutable storage, read only, random access, sequential access, location addressable, file addressable, content addressable, network attached storage, storage area network, bar codes, magnetic ink, and the like.

The methods and systems described herein may transform physical and/or or intangible items from one state to another. The methods and systems described herein may also transform data representing physical and/or intangible items from one state to another.

The elements described and depicted herein, including in flow charts and block diagrams throughout the figures, imply logical boundaries between the elements. However, according to software or hardware engineering practices, the depicted elements and the functions thereof may be implemented on machines through computer executable media having a processor capable of executing program instructions stored thereon as a monolithic software structure, as standalone software modules, or as modules that employ external routines, code, services, and so forth, or any combination of these, and all such implementations may be within the scope of the present disclosure. Examples of such machines may include, but may not be limited to, personal digital assistants, laptops, personal computers, mobile phones, other handheld computing devices, medical equipment, wired or wireless communication devices, transducers, chips, calculators, satellites, tablet PCs, electronic books, gadgets, electronic devices, devices having artificial intelligence, computing devices, networking equipment, servers, routers and the like. Furthermore, the elements depicted in the flow chart and block diagrams or any other logical component may be implemented on a machine capable of executing program instructions. Thus, while the foregoing drawings and descriptions set forth functional aspects of the disclosed systems, no particular arrangement of software for implementing these functional aspects should be inferred from these descriptions unless explicitly stated or otherwise clear from the context. Similarly, it will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

The methods and/or processes described above, and steps associated therewith, may be realized in hardware, software or any combination of hardware and software suitable for a particular application. The hardware may include a general-purpose computer and/or dedicated computing device or specific computing device or particular aspect or component of a specific computing device. The processes may be realized in one or more microprocessors, microcontrollers, embedded microcontrollers, programmable digital signal processors or other programmable device, along with internal and/or external memory. The processes may also, or instead, be embodied in an application specific integrated circuit, a programmable gate array, programmable array logic, or any other device or combination of devices that may be configured to process electronic signals. It will further be appreciated that one or more of the processes may be realized as a computer executable code capable of being executed on a machine-readable medium.

The computer executable code may be created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software, or any other machine capable of executing program instructions.

Thus, in one aspect, methods described above and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. In another aspect, the means for performing the steps associated with the processes described above may include any of the hardware and/or software described above. All such permutations and combinations are intended to fall within the scope of the present disclosure.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

All documents referenced herein are hereby incorporated by reference.

1. An apparatus, comprising:
a sample holder configured to hold or receive a sample during use;
a first chamber operably coupled to the sample holder and configured to receive the sample holder, the first chamber including at least one extraction reagent for extracting nucleic acid from the sample during use to generate a first treated sample, the first chamber and the sample holder collectively defining a common longitudinal axis; and
a second chamber operably coupled to the first chamber and disposed along the longitudinal axis, the second chamber including at least one oligomer for amplifying the nucleic acid to generate a second treated sample.

2-3. (canceled)

4. The apparatus of claim 1, wherein the sample holder includes one of a swab, a pipette and a syringe.

5-7. (canceled)

8. The apparatus of claim 1, wherein the at least one extraction reagent includes a liquid reagent and the first chamber includes a first storage unit to store the liquid reagent.

9. The apparatus of claim 1, wherein the first chamber includes:
a first compartment configured to couple to the sample holder;
a first filter disposed in the first compartment;
a second compartment operably coupled to the first compartment, the second compartment being translatable with respect to the first compartment; and
a first valve or a first membrane disposed within the first compartment, the first valve being configured to permit fluid flow from the first compartment to the second compartment and to block fluid flow from the second compartment to the first compartment,
wherein the second compartment is configured to draw at least a portion of the first treated sample from the first compartment when moved away from the first compartment.

10-11. (canceled)

12. The apparatus of claim 9, wherein the first chamber further includes at least one sample processing control (SPC).

13. (canceled)

14. The apparatus of claim 9, wherein the second chamber includes:
a third compartment operably coupled to the second compartment;
a second filter disposed in the third compartment; and
a second valve or a second membrane disposed between the second compartment and the third compartment, the second valve being configured to permit fluid flow from the second compartment to the third compartment and to block fluid flow from the third compartment to the second compartment,
wherein the second compartment is configured to transport at least a portion of the first treated sample from the second compartment to the third compartment upon moving closer to the first compartment.

15. The apparatus of claim 14, wherein the at least one oligomer is disposed on the second filter or in the third compartment.

16. The apparatus of claim 1, wherein the second chamber includes an interface configured to permit optical interrogation of the second treated sample in the second chamber.

17-62. (canceled)

63. A system, including:
the apparatus of claim 14; and
a device configured to receive the apparatus, the device including:
a motor configured to move the second compartment with respect to the first compartment so as to transport at least one of the sample or the first treated sample;
a heater configured to thermo-cycle the first treated sample in the second chamber so as to amplify the nucleic acid, the heater being further configured to interact with the first chamber to lyse the sample and extract the nucleic acid from the sample;
a light source configured to illuminate the first treated sample in the second chamber;
a detector, in optical communication with the second chamber, the detector being configured to detect the nucleic acid via fluorescence; and
an optical filter disposed before the detector.

64. The system of claim 63, wherein the device further includes:
a memory to store process-executable instructions; and
a processor operably coupled to the memory, wherein upon execution of the processor-executable instructions, the processor configured to:
control the motor to move the second compartment with respect to the first compartment so as to extract the nucleic acid from the sample; and
control the heater to thermo-cycle the first treated sample so as to amplify the nucleic acid via extreme chain reaction (XCR™).

65. The system of claim 64, wherein upon execution of the process-executable instructions, the processor is further configured to:
control the light source to illuminate the second chamber; and
control the detector to detect the nucleic acid via fluorescence in the second treated sample.

66. (canceled)

67. The system of claim 63, wherein the system is configured to detect at least one, at least two, at least three, at least four, at least five, at least six, at least seven, at least eight, at least nine, at least ten, at least fifteen, or at least twenty target analytes in the test sample.

68. (canceled)

69. A method, comprising:
coupling a sample holder to a first chamber, the sample holder having a sample disposed therein, the sample holder and the first chamber having a common longitudinal axis when coupled, and the first chamber including at least one extraction reagent for extracting nucleic acid from the sample; and
transporting at least a portion of the sample to a second chamber operably coupled to the first chamber and disposed along the common longitudinal axis, the second chamber including at least one oligomer for amplifying the nucleic acid.

70-73. (canceled)

74. The method of claim 69, wherein the at least one extraction reagent includes a liquid reagent stored in a first storage unit within the first chamber and method further comprises transporting the liquid reagent from the first storage unit to the sample holder so as to cause the liquid reagent to be in contact with the sample.

75. The method of claim 69, further comprising:
extracting first nucleic acid using a first extraction reagent preloaded in the first chamber and extracting second nucleic acid using a second extraction reagent preloaded in the first chamber.

76. The method of claim 75, further comprising:
amplifying the first nucleic acid using a first oligomer preloaded in the second chamber and amplifying the second nucleic acid using a second oligomer preloaded in the second chamber.

77. The method of claim 69, wherein coupling the sample holder includes disposing the sample holder into a first compartment operably coupled to a second compartment, and the method further comprises:
moving the second compartment away from the first compartment so as to draw at least a portion of the sample from the first compartment to the second compartment through a first valve and a first filter disposed within the first compartment.

78. (canceled)

79. The method of claim 77, further comprising:
placing the first chamber into a device; and
moving, via a motor of the device, the second compartment away from the first compartment.

80. The method of claim 77, wherein transporting the at least a portion of the sample includes:
moving the second compartment closer to the first compartment so as to transport the at least a portion of the sample through a second valve disposed within the second compartment and into the second chamber.

81-102. (canceled)

103. A sample preparation device, comprising:
a sample holder configured to receive a sample;
a first chamber including:
a first compartment to receive the sample holder, the sample holder being configured to be removable from the first compartment and hermetically seal the sample preparation device when placed in the first compartment;
a second compartment operably coupled to the first compartment; and
a first valve disposed within the first compartment, the first valve being configured to permit fluid flow from the first compartment to the second compartment and to block fluid flow from the second compartment to the first compartment, the second compartment being configured to draw at least a portion of the sample from the first compartment to the second compartment upon moving away from the first compartment, the first chamber including at least one extraction reagent preloaded within the first chamber to extract nucleic acid from target microorganism in the sample; and
a second chamber including:
a third compartment operably coupled to the second compartment; and
a second valve disposed between the second compartment and the third compartment, the second valve being configured to permit fluid flow from the second compartment to the third compartment and to block fluid flow from the third compartment to the second compartment, the second compartment being configured to transport at least a portion of the sample from the second compartment to the third compartment upon moving closer to the first compartment, the second chamber including at least one oligomer preloaded within the second chamber to amplify the nucleic acid extracted by the at least one extraction reagent.
